(19) United States
(12) Patent Application Publication
(10) Pub. No.: US 2014/0256698 A1
(43) Pub. Date: Sep. 11, 2014

(54) CATHEPSIN INHIBITORS

(71) Applicant: ViroBay, Inc., Menlo Park, CA (US)

(72) Inventors: Robert Booth, Los Altos Hills, CA (US); Jeff Dener, Millbrae, CA (US); Michael Green, Haif Moon Bay, CA (US)

(21) Appl. No.: 14/205,087

(22) Filed: Mar. 11, 2014

Related U.S. Application Data
(60) Provisional application No. 61/776,646, filed on Mar. 11, 2013.

(51) Int. Cl. C07C 237/22; C07C 253/30; C07C 255/29; C07C 231/12; C07D 211/14; C07D 257/04; C07D 207/06; C07D 295/155; C07D 307/16; C07D 205/04; C07D 233/61; C07D 231/12; C07C 255/46; C07D 309/06

(52) U.S. Cl. CPC C07C 237/22; C07C 255/46; C07C 253/30; C07C 255/29; C07C 231/12; C07D 211/14; C07D 309/06; C07D 207/06; C07D 295/155; C07D 307/16; C07D 205/04; C07D 233/61; C07D 231/12; C07D 257/04
USPC 514/210.01; 558/426; 514/620; 558/393; 564/157; 514/616; 546/234; 514/331; 549/426; 514/459; 548/568; 514/428; 544/168; 514/237.8

(57) ABSTRACT

The present invention is directed to inhibitors of cathepsins and the methods for using and making such inhibitors.

### CROSS REFERENCE TO RELATED APPLICATION

The instant patent application claims priority to U.S. Provisional Patent Application Ser. No. 61/776,646, filed Mar. 11, 2013, the entire contents of which are herein incorporated by reference for all purposes.

### BACKGROUND OF THE INVENTION

The present invention is directed to inhibitors of cathepsins and the methods for using and making such inhibitors.

Cysteine proteases such as cathepsins B, H, K, L, O and S, represent a class of peptidases characterized by the presence of a cysteine residue in the catalytic site of the enzyme. Cysteine proteases are associated with the normal degradation and processing of proteins. The aberrant activity of cysteine proteases, e.g., as a result of increased expression or enhanced activation, however, has pathological consequences. In this regard, certain cysteine proteases are associated with a number of disease states, including arthritis, muscular dystrophy, inflammation, tumor invasion, glomerulonephritis, periodontal disease, and metachromatic leukodystrophy. For example, increased cathepsin B levels and redistribution of the enzyme are found in tumors, thus demonstrating a role for the enzyme in tumor invasion and metastasis. In addition, aberrant cathepsin B activity is implicated in such disease states as Alzheimer's Disease, arthritis, inflammatory diseases such as chronic and acute pancreatitis, inflammatory airway disease, and bone and joint disorders, including osteoporosis, osteoarthritis, rheumatoid arthritis, psoriasis, and other autoimmune disorders.

Cathepsin B is also associated with fibrotic disease, including HCV-associated liver fibrosis, all types of steatosis (including non-alcoholic steatohepatitis) and alcohol-associated steatohepatitis, non-alcoholic fatty liver disease, forms of pulmonary fibrosis including idiopathic pulmonary fibrosis, pathological diagnosis of interstitial pneumonia following lung biopsy, renal fibrosis, cardiac fibrosis, retinal angiogenesis and fibrosis/gliosis in the eye, scleroderma, systemic sclerosis, and keloids and other forms of scarring.

In view of the number of diseases or conditions related to the normal activity or the increased expression of cathepsin B, compounds that are capable of inhibiting enzymatic protease activity or expression would accordingly be useful.

### SUMMARY OF THE INVENTION

An aspect of this invention is a compound of Formula I:

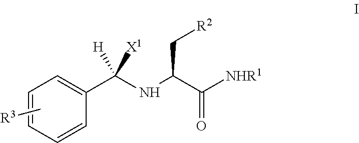

in which:

### X¹ is —CF₃, —CF₂CF₃ or —CHF₂;

R¹ is a group of Formula (a), (b) or (c):

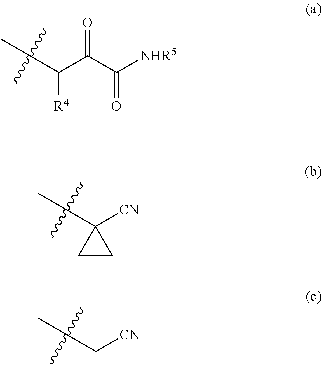

where R⁴ is (C₁₋₃)-n-alkyl or (C₃₋₄)cycloalkylmethyl and R⁵ is hydrogen, (C₁₋₃)alkyl or (C₃₋₇)cycloalkyl;
R² is a group of Formula (d), (e) or (f):

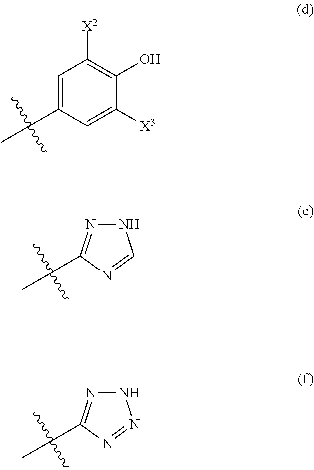

where X² and X³ are independently iodo, bromo, fluoro or chloro; and
R³ is one to three substituents selected from hydrogen, C₁₋₆alkoxy, C₃₋₆cycloalkoxy, fluoro, chloro, bromo, trifluoromethyl, trifluoromethoxy, heteroaryl or heterocyclyl, wherein the heteroaryl and heterocyclyl may be further substituted with 2,2,2-trifluoroethyl, (C₁₋₆)alkyl, or (C₃₋₆)cycloalkyl; and the pharmaceutically acceptable salts thereof.

A second aspect of this invention is a pharmaceutical composition comprising a compound of this invention or a pharmaceutically acceptable salt thereof and a pharmaceutically acceptable excipient.

A third aspect of this invention is a method for treating a disease in an animal mediated by cysteine proteases, in particular cathepsin B, which method comprises administering to the animal a pharmaceutical composition comprising a therapeutically effective amount of a compound of the invention or a pharmaceutically acceptable salt thereof and a pharmaceutically acceptable excipient.

A fourth aspect of this invention is directed to processes for preparing compounds described herein and pharmaceutically acceptable salts thereof.

A fifth aspect is a process for preparing a compound of the invention, e.g., a compound of Formula (I), wherein R¹ is a group of Formula (a), (b) or (c), which process comprises contacting a compound of Formula 4:

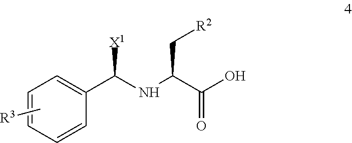

with a compound having the formula NH₂R⁶, where R⁶ is group of Formula (a), (b) or (c), in the presence of a suitable coupling agent and base, wherein the compound of Formula 4 is prepared by reducing the compound of Formula 3:

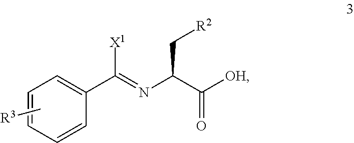

wherein the compound of Formula 3 is prepared by contacting a compound of Formula 1:

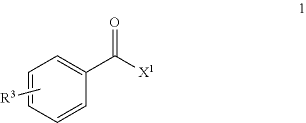

with a compound of Formula 2:

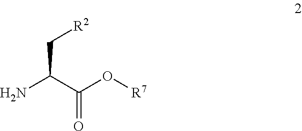

in which R⁷ is C₁₋₇alkyl or C₃₋₆cycloalkylmethyl, in the presence of a weak base;
in which X¹, R¹, R², R³, R⁴, R⁵ and R⁶ are as defined in the Summary of the Invention.

### DETAILED DESCRIPTION OF THE INVENTION

### Definitions

Unless otherwise stated, the following terms used in the specification and claims are defined for the purposes of this application and have the following meanings.

“Alkyl” represented by itself means a straight or branched, saturated aliphatic radical containing one to six carbon atoms, unless otherwise indicated, e.g., alkyl includes methyl, ethyl, propyl, isopropyl, butyl, sec-butyl, isobutyl, tert-butyl, and the like.

“Animal” includes humans, non-human mammals (e.g., dogs, cats, rabbits, cattle, horses, sheep, goats, swine, deer, and the like) and non-mammals (e.g., birds, and the like).

“Aromatic” refers to a moiety wherein the constituent atoms make up an unsaturated ring system, all atoms in the ring system are sp² hybridized and the total number of pi electrons is equal to 4n+2.

“Aryl” refers to a monocyclic or fused bicyclic ring assembly containing 6 to 10 ring carbon atoms wherein each ring is aromatic, e.g., phenyl or naphthyl.

“Cycloalkyl” refers to a monovalent saturated or partially unsaturated, monocyclic ring containing three to eight ring carbon atoms, e.g., cyclopropyl, cyclobutyl, cyclopentyl, cyclohexyl, cyclohexenyl, 2,5-cyclohexadienyl, and the like.

“Disease” specifically includes any unhealthy condition of an animal or part thereof and includes an unhealthy condition that may be caused by, or incident to, medical or veterinary therapy applied to that animal, i.e., the “side effects” of such therapy.

“Halo” refers to fluoro, chloro, bromo or iodo.

“Heteroaryl” as a group or part of a group denotes an aromatic monocyclic or multicyclic moiety of 5 to 10 ring atoms in which one or more, preferably one, two, or three, of the ring atom(s) is (are) selected from nitrogen, oxygen or sulfur, the remaining ring atoms being carbon. Representative heteroaryl rings include, but are not limited to, pyrrolyl, furanyl, thienyl, oxazolyl, isoxazolyl, thiazolyl, imidazolyl, triazolyl, tetrazolyl, pyridinyl, pyrimidinyl, pyrazinyl, pyridazinyl, indolyl, benzofuranyl, benzothienyl, benzimidazolyl, quinolinyl, isoquinolinyl, quinazolinyl, quinoxalinyl, pyrazolyl, and the like.

“Heterocyclyl” refers to a saturated or partially unsaturated, mono or bicyclic radical of 4, 5 or 6 carbon ring atoms wherein one or more, preferably one, two, or three of the ring carbon atoms are replaced by a heteroatom selected from —N═, —N—, —O—, —S—, —SO—, or —S(O)₂— and further wherein one or two ring atoms are optionally replaced by a keto (—CO—) group. The heterocyclyl ring is optionally fused to aryl or heteroaryl ring as defined herein. Representative examples include, but are not limited to, imidazolidinyl, morpholinyl, thiomorpholinyl, thiomorpholino-1-oxide, thiomorpholino-1,1-dioxide, tetrahydropyranyl, tetrahydrothiopyranyl, 1-oxo-tetrahydrothiopyranyl, 1,1-dioxotetrathiopyranyl, indolinyl, piperazinyl, piperidyl, pyrrolidinyl, pyrrolinyl, quinuclidinyl, and the like.

“Hydroxy” means —OH radical.

“Isomers” mean compounds of the invention having identical molecular formulae but differing in the nature or sequence of bonding of their atoms or in the arrangement of their atoms in space. Isomers that differ in the arrangement of their atoms in space are termed “stereoisomers.” Stereoisomers that are not mirror images of one another are termed “diastereomers,” and stereoisomers that are nonsuperimposable mirror images are termed “enantiomers” or sometimes “optical isomers.” A carbon atom bonded to four nonidentical substituents is termed a “chiral center.” A compound with one chiral center has two enantiomeric forms of opposite chirality is termed a “racemic mixture.” A compound that has more than one chiral center has 2n⁻¹ enantiomeric pairs, where n is the number of chiral centers. Compounds with more than one chiral center may exist as either an individual diastereomer or as a mixture of diastereomers, termed a “diastereomeric mixture.” When one chiral center is present a stereoisomer may be characterized by the absolute configuration of that chiral center. Absolute configuration refers to the arrangement in space of the substituents attached to the chiral center. Enantiomers are characterized by the absolute configuration of their chiral centers and described by the R- and S-sequencing rules of Cahn, Ingold and Prelog. Conventions for stereochemical nomenclature, methods for the determination of stereochemistry and the separation of stereoisomers are well known in the art (e.g., see “Advanced Organic Chemistry”, 4th edition, March, Jerry, John Wiley & Sons, New York, 1992). It is understood that the names and illustration used in this application to describe compounds of the invention are meant to be encompassed all possible stereoisomers.

“Optional” or “optionally” or “may be” means that the subsequently described event or circumstance may or may not occur, and that the description includes instances where the event or circumstance occurs and instances in which it does not.

The present invention also includes N-oxide derivatives of a compound of the invention. N-oxide derivative mean a compound of the invention in which a nitrogen atom is in an oxidized state (i.e., N→O) e.g., pyridine N-oxide, and which possess the desired pharmacological activity.

“Pathology” of a disease means the essential nature, causes and development of the disease as well as the structural and functional changes that result from the disease processes.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of compounds of the invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methylsulfonic acid, ethanesulfonic acid, 1,2-ethanedisulfonic acid, 2-hydroxy-ethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like.

Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

The present invention also includes prodrugs of a compound of the invention. Prodrug means a compound that is convertible in vivo by metabolic means (e.g., by hydrolysis) to a compound of the invention. For example an ester of a compound of the invention containing a hydroxy group may be convertible by hydrolysis in vivo to the parent molecule. Alternatively an ester of a compound of the invention containing a carboxy group may be convertible by hydrolysis in vivo to the parent molecule. Suitable esters of compounds of the invention containing a hydroxy group, are for example acetates, citrates, lactates, tartrates, malonates, oxalates, salicylates, propionates, succinates, fumarates, maleates, methylene-bis-b-hydroxynaphthoates, gentisates, isethionates, di-p-toluoyltartrates, methylsulphonates, ethanesulphonates, benzenesulphonates, p-toluenesulphonates, cyclohexylsulphamates and quinates. Suitable esters of compounds of the invention containing a carboxy group, are for example those described by Leinweber, F. J. Drug Metab. Res., 1987, 18, page 379. An especially useful class of esters of compounds of the invention containing a hydroxy group, may be formed from acid moieties selected from those described by Bundgaard et al., J. Med. Chem., 1989, 32, pp 2503-2507, and include substituted (aminomethyl)-benzoates, for example, dialkylamino-methylbenzoates in which the two alkyl groups may be joined together and/or interrupted by an oxygen atom or by an optionally substituted nitrogen atom, e.g., an alkylated nitrogen atom, more especially (morpholino-methyl)benzoates, e.g., 3- or 4-(morpholinomethyl)-benzoates, and (4-alkylpiperazin-1-yl)benzoates, e.g., 3- or 4-(4-alkylpiperazin-1-yl)benzoates.

“Protected derivatives” means derivatives of compounds of the invention in which a reactive site or sites are blocked with protecting groups. Protected derivatives of compounds of the invention are useful in the preparation of compounds of the invention or in themselves may be active cathepsin S inhibitors. A comprehensive list of suitable protecting groups can be found in T. W. Greene, Protective Groups in Organic Synthesis, 3rd edition, John Wiley & Sons, Inc. 1999.

“Therapeutically effective amount” means that amount which, when administered to an animal for treating a disease, is sufficient to effect such treatment for the disease.

“Treatment” or “treating” means any administration of a compound of the present invention and includes:

(1) preventing the disease from occurring in an animal which may be predisposed to the disease but does not yet experience or display the pathology or symptomatology of the disease,
(2) inhibiting the disease in an animal that is experiencing or displaying the pathology or symptomatology of the diseased (i.e., arresting further development of the pathology and/or symptomatology), or
(3) ameliorating the disease in an animal that is experiencing or displaying the pathology or symptomatology of the diseased (i.e., reversing the pathology and/or symptomatology).

Certain compounds of the invention within the broadest scope set forth in the Summary of the Invention are preferred.

Preferred are compounds of the invention in which R¹ is a group of Formula (b) or (c):

Preferred compounds of the invention are:
N-cyanomethyl-2S-[2,2,2-trifluoro-1S-(4-bromophenyl)-ethylamino]-3-(3,5-diiodo-4-hydroxyphenyl)-propanamide;
N-(1-cyanocyclopropyl)-2S-[2,2,2-trifluoro-1S-(4-bromophenyl)-ethylamino]-3-(3,5-diiodo-4-hydroxyphenyl)-propanamide;
N-(1-cyanocyclopropyl)-2S-[2,2,2-trifluoro-1S-(4-bromophenyl)-ethylamino]-3-(3,5-dichloro-4-hydroxyphenyl)-propanamide; and
N-(1-cyanocyclopropyl)-2S-[2,2,2-trifluoro-1S-(4-fluorophenyl)-ethylamino]-3-(3,5-dichloro-4-hydroxyphenyl)-propanamide.

In some embodiments, the compound of Formula (I) is N-cyanomethyl-2S-[2,2,2-trifluoro-1S-(4-bromophenyl)-ethylamino]-3-(3,5-diiodo-4-hydroxyphenyl)-propanamide. In certain other embodiments, the compound of Formula (I) is N-(1-cyanocyclopropyl)-2S—[2,2,2-trifluoro-1S-(4-bromophenyl)-ethylamino]-3-(3,5-diiodo-4-hydroxyphenyl)-propanamide. In yet other embodiments, the compound of Formula (I) is N-(1-cyanocyclopropyl)-2S-[2,2,2-trifluoro-1S-(4-bromophenyl)-ethylamino]-3-(3,5-dichloro-4-hydroxyphenyl)-propanamide. In other embodiments, the compound of Formula (I) is N-(1-cyanocyclopropyl)-2S-[2,2,2-trifluoro-1S-(4-fluorophenyl)-ethylamino]-3-(3,5-dichloro-4-hydroxyphenyl)-propanamide. In certain other embodiments, the compound of Formula (I) is N-cyclopropyl-3S-{3-[3,5-diiodo-4-hydroxyphenyl-2S-(2,2,2-trifluoro)-1S-(4-bromophenyl)ethylamino]propionylamino}-2-oxopentanamide. In other embodiments, the compound of Formula (I) is N-cyclopropyl-3S-{3-[3,5-diiodo-4-hydroxyphenyl-2S-(2,2,2-trifluoro)-1S-(4-fluorophenyl)ethylamino]propionylamino}-2-oxopentanamide. In some other embodiments, the compound of Formula (I) is N-cyclopropyl-3S-{3-[3,5-dichloro-4-hydroxyphenyl-2S-(2,2,2-trifluoro)-1S-(4-fluorophenyl)ethylamino]propionylamino}-2-oxopentanamide. In some embodiments, the compound of Formula (I) is N-cyclopropyl-3S-{3-[3,5-dichloro-4-hydroxyphenyl-2S-(2,2,2-trifluoro)-1S-(4-bromophenyl)ethylamino]propionylamino}-2-oxopentanamide.

Further preferred are compounds of the invention in which R¹ is a group of Formula (a).

Further preferred compounds of the invention are:
N-cyclopropyl-3S-{3-[3,5-diiodo-4-hydroxyphenyl)-2S-(2,2,2-trifluoro)-1S-(4-bromophenyl)ethylamino]propionylamino}-2-oxopentanamide;
N-cyclopropyl-3S-{3-[3,5-diiodo-4-hydroxyphenyl)-2S-(2,2,2-trifluoro)-1S-(4-fluorophenyl)ethylamino]propionylamino}-2-oxopentanamide;
N-cyclopropyl-3S-{3-[3,5-dichloro-4-hydroxyphenyl)-2S-(2,2,2-trifluoro)-1S-(4-fluorophenyl)ethylamino]propionylamino}-2-oxopentanamide; and
N-cyclopropyl-3S-{3-[3,5-dichloro-4-hydroxyphenyl)-2S-(2,2,2-trifluoro)-1S-(4-bromophenyl)ethylamino]propionylamino}-2-oxopentanamide.

In some embodiments, the compound described herein is N-cyanomethyl-2S-[2,2,2-trifluoro-1S-(4-bromophenyl)-ethylamino]-3-(3,5-diiodo-4-hydroxyphenyl)-propanamide. In other embodiments, the compound described herein is N-(1-cyanocyclopropyl)-2S-[2,2,2-trifluoro-1S-(4-bromophenyl)-ethylamino]-3-(3,5-diiodo-4-hydroxyphenyl)-propanamide. In yet other embodiments, the compound described herein includes N-(1-cyanocyclopropyl)-2S—[2,2,2-trifluoro-1S-(4-bromophenyl)-ethylamino]-3-(3,5-dichloro-4-hydroxyphenyl)-propanamide. In still other embodiments, the compound described herein includes N-(1-cyanocyclopropyl)-2S-[2,2,2-trifluoro-1S-(4-fluorophenyl)-ethylamino]-3-(3,5-dichloro-4-hydroxyphenyl)-propanamide.

In yet other embodiments of the invention, the compound described herein includes a compound of the invention in which R¹ is a group of Formula (a). In other embodiments of the invention, the compound described herein include a compound in which R¹ is a group of Formula (b). In other embodiments of the invention, the compound described herein include a compound in which R¹ is a group of Formula (b).

Further preferred compounds of the invention are:
N-cyclopropyl-3S-{3-[3,5-diiodo-4-hydroxyphenyl)-2S-(2,2,2-trifluoro)-1S-(4-bromophenyl)ethylamino]propionylamino}-2-oxopentanamide;
N-cyclopropyl-3S-{3-[3,5-diiodo-4-hydroxyphenyl)-2S-(2,2,2-trifluoro)-1S-(4-fluorophenyl)ethylamino]propionylamino}-2-oxopentanamide;
N-cyclopropyl-3S-{3-[3,5-dichloro-4-hydroxyphenyl)-2S-(2,2,2-trifluoro)-1S-(4-fluorophenyl)ethylamino]propionylamino}-2-oxopentanamide; and
N-cyclopropyl-3S-{3-[3,5-dichloro-4-hydroxyphenyl)-2S-(2,2,2-trifluoro)-1S-(4-bromophenyl)ethylamino]propionylamino}-2-oxopentanamide.

The compounds of the present invention include those compounds within the scope defined by Formula I:

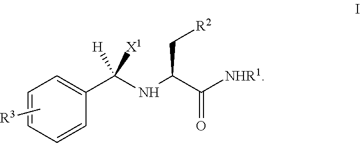

Some compounds of Formula (I) include those compounds wherein X is CF₃. Other compounds of Formula (I) include those compounds wherein X is CF₂CF₃. Some other compounds of Formula (I) include those compounds where X is CHF₂.

Certain compounds of Formula (I) include those compounds wherein R¹ is Formula (a). Other compounds of Formula (I) include those compounds wherein R¹ is Formula (b). Some other compounds of Formula (I) include those compounds wherein R¹ is Formula (c).

In any of the above described compounds, wherein R³ is CF₃, R¹ includes Formula (a). In any of the above described compounds, wherein R³ is CF₃, R¹ includes Formula (b). In any of the above described compounds, wherein R³ is CF₃, R¹ includes Formula (c).

In some embodiments, the compounds of Formula (I) include those where R³ is bromo. Certain of these compounds include R³ as bromo at the para position of the phenyl ring. Certain other of these compounds include R³ as bromo at the meta position of the phenyl ring. Some other of these compounds include R³ as bromo at the ortho position of the phenyl ring.

In some embodiments, the compounds of Formula (I) include those where R³ is fluoro. Certain of these compounds include R³ as fluoro at the ortho position of the phenyl ring. Certain other of these compounds include R³ as fluoro at the meta position of the phenyl ring. Some other of these compounds include R³ as fluoro at the para position of the phenyl ring.

In some embodiments, the compounds of Formula (I) include those where R³ is chloro. Certain of these compounds include R³ as chloro at the para position of the phenyl ring. Some of these compounds include R³ as chloro at the meta position of the phenyl ring. Certain other of these compounds include R³ as chloro at the ortho position of the phenyl ring.

In some other embodiments, the compounds of Formula (I) include those where R³ is pyridinyl. Certain of these compounds include R³ as pyridinyl at the para position of the phenyl ring. In some of these compounds, the R³ substituent is attached to the phenyl ring at the 2-position. In some other embodiments, the R³ substituent is attached to the phenyl ring at the 3-position.

In some embodiments, the compounds of Formula (I) include those where R³ is CF₃O. Certain of these compounds include R³ as CF₃O at the para position of the phenyl ring.

In some embodiments, the compounds of Formula (I) include compounds wherein R³ is

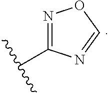 [SMILES: *c1ncon1]

In some of these compounds, R³ is at the para position of the phenyl ring.

In some embodiments, the compounds of Formula (I) include compounds wherein R³ is

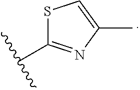 [SMILES: *c1nc(C)cs1]

In some of these compounds, R³ is at the meta position of the phenyl ring.

In the compounds described herein, the symbol,

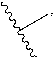

represents the point of attachment of a substituent to the remainder of the compound.

In some embodiments, the compounds of Formula (I) include compounds wherein R³ is

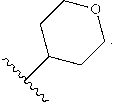 [SMILES: *C1CCOCC1]

In some of these compounds, R³ is at the para position of the phenyl ring. In some of these compounds, R³ is at the para position of the phenyl ring.

In some embodiments, the compounds of Formula (I) include compounds wherein R³ is

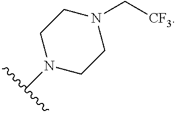 [SMILES: *N1CCN(CC(F)(F)F)CC1]

In some other embodiments, the compounds of Formula (I) include compounds wherein R³ is

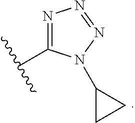 [SMILES: *c1nnnn1C1CC1]

In some of the compounds of Formula (I) described above, R⁴ is ethyl. In some other of the compounds of Formula (I) described above, R⁴ is propyl. In some of the compounds of Formula (I) described above, R⁴ is cyclopropylmethyl. In some of the compounds of Formula (I) described above, R⁴ is cyclobutylmethyl.

In some of the embodiments of the compounds of Formula (I) described herein and above, R² is

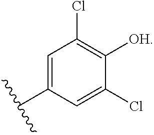 [SMILES: *c1cc(Cl)c(O)c(Cl)c1]

In some other embodiments of the compounds of Formula (I) described herein and above, R² is

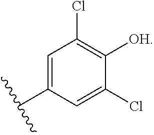 [SMILES: *c1cc(Cl)c(O)c(Cl)c1]

In some of the embodiments of the compounds of Formula (I) described herein and above, R² is

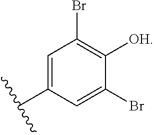 [SMILES: *c1cc(Br)c(O)c(Br)c1]

In certain of the embodiments of the compounds of Formula (I) described herein and above, R² is

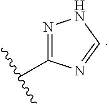 [SMILES: *c1nc[nH]n1]

In some other of the embodiments of the compounds of Formula (I) described herein and above, R² is

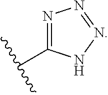 [SMILES: *c1nnn[nH]1]

In some of the embodiments of the compounds of Formula (I), R¹ is Formula (a):

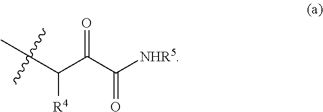

In some of the embodiments of the compounds of Formula (I), R¹ is Formula (b):

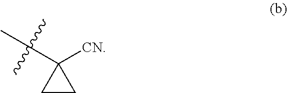 [SMILES: *C1(C#N)CC1]

In some of the embodiments of the compounds of Formula (I), R¹ is Formula (c):

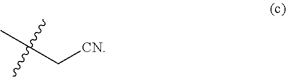 [SMILES: *CC#N]

In some of the embodiments of the compounds of Formula (I), R⁴ is (C₁₋₃)-n-alkyl. In some other of the embodiments of the compounds of Formula (I), R⁴ is (C₃₋₄)cycloalkylmethyl.

In some of the embodiments of the compounds of Formula (I), R⁵ is hydrogen. In other embodiments of the compounds of Formula (I), R⁵ is (C₁₋₃)alkyl. In still others, or the compounds of Formula (I) include those compounds wherein R⁵ is (C₃₋₇)cycloalkyl.

In some of the embodiments of the compounds of Formula (I), R² is a group of Formula (d):

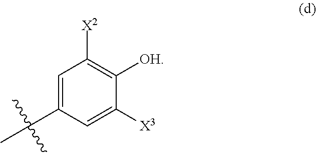

In some other of the embodiments of the compounds of Formula (I), R² is a group of (e):

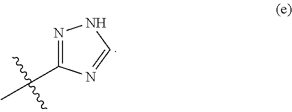 [SMILES: *c1nc[nH]n1]

In certain other embodiments of the compounds of Formula (I), R² is a group of (f):

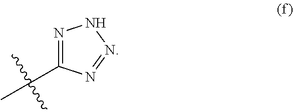 [SMILES: *c1nn[nH]n1]

In some of the embodiments of the compounds of Formula (I), X² is fluoro. In some other of the embodiments of the compounds of Formula (I), X² is bromo. In yet some of the embodiments of the compounds of Formula (I), X² is chloro. In other embodiments of the compounds of Formula (I), X² is iodo.

In some of the embodiments of the compounds of Formula (I), X³ is fluoro. In some other of the embodiments of the compounds of Formula (I), X³ is bromo. In yet some of the embodiments of the compounds of Formula (I), X³ is chloro. In other embodiments of the compounds of Formula (I), X³ is iodo.

In certain of the above described compounds of Formula (I), R³ is one to three substituents selected from hydrogen. In some other of the above described compounds of Formula (I), R³ is one to three substituents selected from C₁₋₆alkoxy. In other embodiments, the compounds of Formula (I) include those compounds where R³ is C₃₋₆cycloalkoxy. In other embodiments, R³ is fluoro. In certain other embodiments, the compounds of Formula (I) include those compounds wherein R³ is chloro. In some other embodiments, the compounds of Formula (I) include those compounds wherein R³ is chloro. In other embodiments, the compounds of Formula (I) include those compounds wherein R³ is bromo. In certain other embodiments, the compounds of Formula (I) include those compounds wherein R³ is trifluoromethyl. In some other embodiments, the compounds of Formula (I) include those compounds wherein R³ is trifluoromethoxy.

In yet other embodiments, the compounds of Formula (I) include those compounds wherein R³ is heteroaryl. In certain other embodiments, the compounds of Formula (I) include those compounds wherein R³ is heterocyclyl. In these embodiments, the heteroaryl and heterocyclyl may be further substituted with 2,2,2-trifluoroethyl, (C₁₋₆)alkyl, or (C₃₋₆)cycloalkyl.

The present application also sets forth compositions that include a compound described herein or a pharmaceutically acceptable salts thereof.

General Synthetic Scheme

Compounds of this invention in which R¹ is a group of Formula (b) are prepared by proceeding as in Scheme 1:

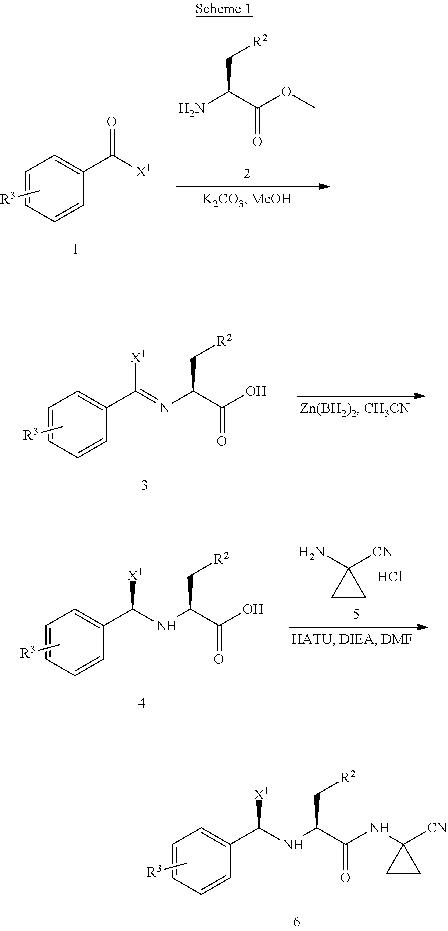

in which X¹, R² and R³ are as defined in the Summary of the Invention.

In general, compounds of Formula 3 are prepared by reacting a compound of Formula 1 with a compound of Formula 2. This reaction is carried out in the presence of a weak base, e.g., potassium carbonate, and in a suitable solvent, e.g., methanol, at approximately 50° C. and requires about 8 hours to complete. Compounds of Formula 4 are prepared by the reduction of a compound of Formula 3. The reduction is carried out with a suitable reducing agent, e.g., zinc borohydride, in the presence in suitable solvent, e.g., acetonitrile and/or THF, at about −40° C. and requires approximately 4 hours to complete. Compounds of Formula 6 are prepared by reacting a compound of Formula 4 with compound of Formula 5. This reaction is carried out in the presence in a suitable coupling agent, e.g., HATU, and base, e.g., diisopropylethylamine, and in a suitable solvent, e.g., DMF. Compounds of the invention where R¹ is a group of Formula (c) are made by proceeding as in Scheme 1, but replacing the compound of Formula 5 with a compound of the formula NH₂CH₂CN.

Alternatively, compounds of Formula 4 wherein R² is a group of Formula (d) where X² and X³ are both chloro can be prepared by proceeding as in Scheme 2:

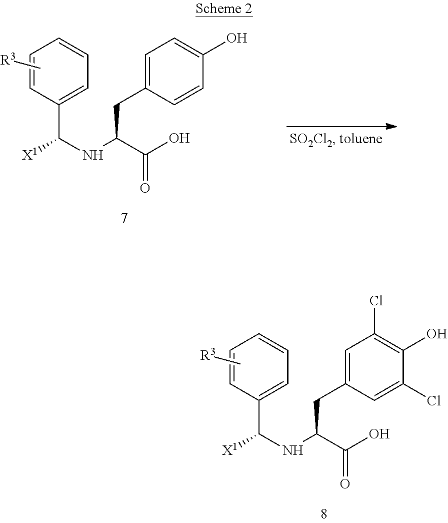

where X¹ and R³ are as defined in the Summary of the Invention.

Compounds of Formula 8 are prepared by treating a compound of Formula 7 with sulfuryl chloride. The reaction is carried out with about 3 equivalents of the sulfuryl chloride in a suitable solvent, e.g., toluene, at about 80° C. and requires approximately 3 hours to complete. Compounds of Formula 4 wherein R² is 3,5-diiodo-4-hydroxyphenyl are prepared by proceeding as in Scheme 2 but replacing sulfuryl chloride with iodine chloride. The reaction is carried out with 2-3 equivalents of the iodine chloride in a suitable solvent, e.g., acetic acid, at about ambient temperature and requires approximately 72 hours to complete. Compounds of Formula 7 are prepared by proceeding as in Scheme 1 but substituting the compound of Formula 2 with methyl 3-(4-hydroxyphenyl)-2-aminoproprionate.

Compounds of this invention in which R¹ is a group of Formula (a) are prepared by proceeding as in Scheme 3:

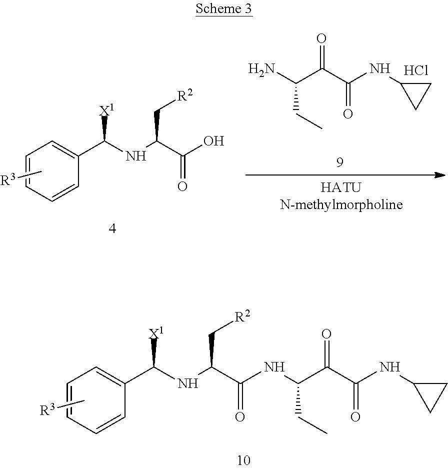

in which X¹, R² and R³ are as defined in the Summary of the Invention.

Compounds of Formula 10 can be prepared by reacting a compound of Formula 4 with a compound of Formula 9. This reaction is carried out in the presence in a suitable coupling agent, e.g., HATU, and base, e.g., diisopropylethylamine, and in a suitable solvent, e.g., DMF. The compound of Formula 9 is prepared by protecting 3S-amino-N-cyclopropyl-2-hydroxypentanamide hydrochloride to give 3S-tert-butoxycarbonylamino-N-cyclopropyl-2-hydroxypentanamide hydrochloride, reducing the 3S-tert-butoxycarbonylamino-N-cyclopropyl-2-hydroxypentanamide hydrochloride to give 3S-tert-butoxycarbonylamino-N-cyclopropyl-2-oxopentanamide and then deprotecting to give 3S-amino-N-cyclopropyl-2-oxopentanamide hydrochloride. The protection step is carried out by treatment with di-tert-butyl dicarbonate in a suitable solvent, e.g., THF and saturated NaHCO₃, at ambient temperature and requires 5 to 6 hours to complete. The oxidation step can be carried out with a suitable oxidizing agent, e.g., Dess-Martin reagent, in a suitable solvent, e.g., dichloromethane, at ambient temperature and requires 3 to 4 hours to complete. The deprotection step can be carried out with acid, e.g., HCl, in a suitable solvent, e.g., isopropanol, at 40 to 50° C. and requires 40 to 60 minutes to complete.

Compounds of this invention in which R¹ is a group of Formula (b) and R² is a group of Formula (f) are prepared by proceeding as in Scheme 4:

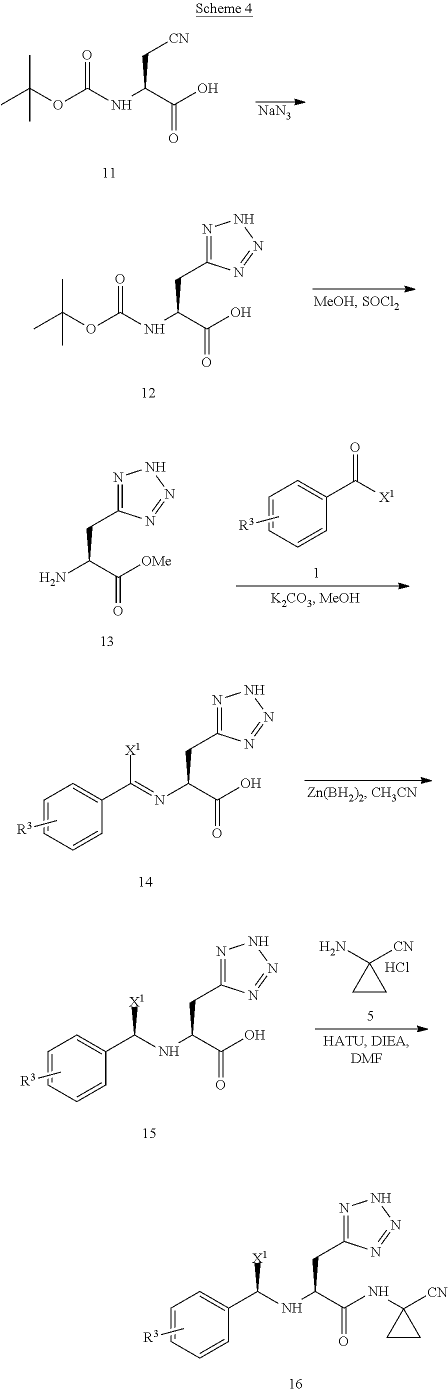

where X¹ and R³ is as defined in the Summary of the Invention.

The compound of Formula 12 is prepared by treating the compound of Formula 11 with sodium azide in a suitable solvent at temperature and requires 1-2 hours to complete. Compounds of Formula 13 are prepared by reacting a compound of Formula 12 with thionyl chloride in methanol and requires 1-2 hours to complete. Compounds of Formula 14 can be prepared by reacting a compound of Formula 13 in the presence of a weak base, e.g., potassium carbonate and in a suitable solvent, e.g., methanol, at approximately 50° C. and requires about 8 hours to complete. The compound of Formula 15 is prepared by reacting the compound of Formula 14 with a suitable reducing agent, e.g., zinc borohydride, in a suitable solvent,e.g., acetonitrile and/or THF, at about −40° C. and requires approximately 4 hours to complete. The compound of Formula 16 is prepared by reacting a compound of Formula 15 in the presence in a suitable coupling agent, e.g., HATU, and base, e.g., diisopropylethylamine, and in a suitable solvent, e.g., DMF.

Compounds of Formula 2 where R² is a group of Formula (e) can be prepared proceeding as in reaction Scheme 5:

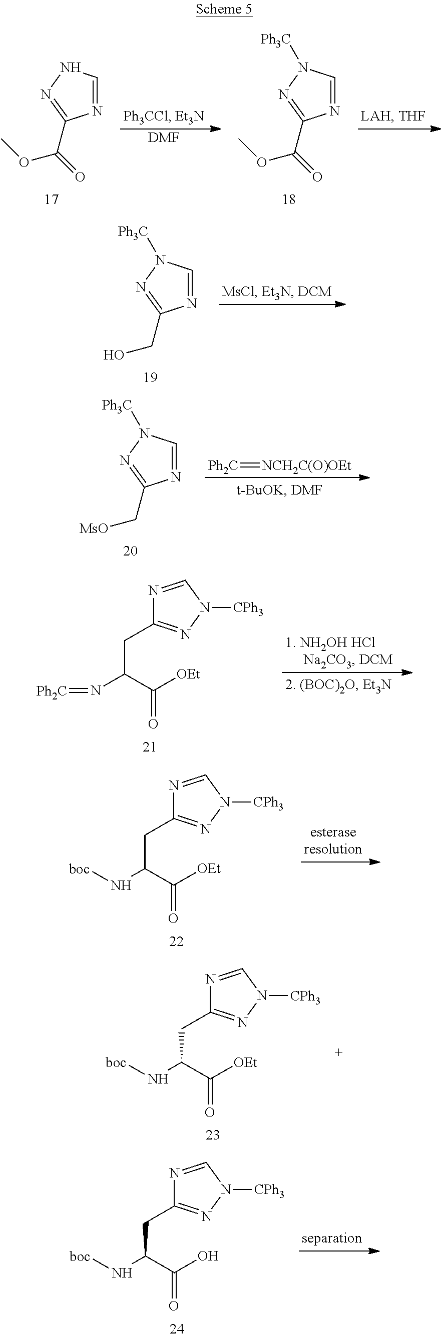

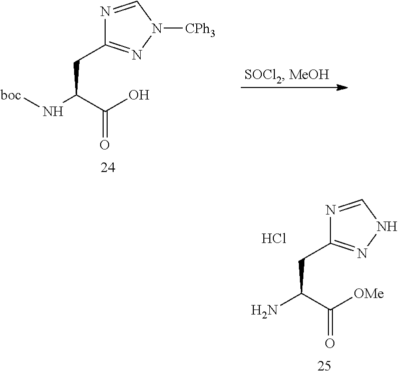

The compound of Formula 18 is prepared by reacting the compound of Formula 17 with trityl chloride in the presence of triethylamine in DMF. The reaction is carried out at ambient temperature. The compound of Formula 19 is prepared by reacting the compound of Formula 18 with a suitable reducing agent, e.g., LAH, in a suitable solvent, e.g., THF, at 0° C. The compound of Formula 20 is prepared by reacting the compound of Formula 19 with mesyl chloride in the presence of base, e.g., triethylamine, in a suitable solvent, e.g., DCM, at about −30° C. The compound of Formula 21 is prepared by treating ethyl 2-diphenylmethyliminoacetate with potassium tert-butoxide in a suitable solvent, e.g., DMF, and then reacting with the compound of Formula 20. The compound of Formula 22 is prepared by reacting the compound of Formula 21 first with hydroxylamine hydrochloride and sodium carbonate in a suitable solvent, e.g., DCM, and second treating the mixture with di-tert-butyl dicarbonate and triethylamine. The compound of Formula 22 is then resolved into its two epimers by treatment with an esterase such as alkylase to afford a mixture of compounds of Formulae 23 and 24. The compound of Formula 25 is prepared by isolating the compound of Formula 24 and then reacting with thionyl chloride in methanol. Alternatively, the resolution of the compound of Formula 22 into its two epimers can be achieved by passing the mixture through a chiral column.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure.

The starting materials and the intermediates of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., more preferably from about 0° C. to about 125° C. and most preferably at about ambient temperature, e.g., about 20° C.

In the reactions described hereinafter it may be necessary to protect reactive functional groups, for example hydroxy, amino, imino, thio or carboxy groups, where these are desired in the final product, to avoid their unwanted participation in the reactions. Conventional protecting groups may be used in accordance with standard practice, for examples see T.W. Greene and P.G.M. Wuts in “Protective Groups in Organic Chemistry” John Wiley and Sons, 1999.

The N-oxides of compounds of the invention can be prepared by methods known to those of ordinary skill in the art. For example, N-oxides can be prepared by treating an unoxidized form of the compound of the invention with an oxidizing agent (e.g., trifluoroperacetic acid, permaleic acid, perbenzoic acid, peracetic acid, meta-chloroperoxybenzoic acid, or the like) in a suitable inert organic solvent (e.g., a halogenated hydrocarbon such as dichloromethane) at approximately 0° C. Alternatively, the N-oxides of the compounds of the invention can be prepared from the N-oxide of an appropriate starting material.

Compounds of the invention in unoxidized form can be prepared from N-oxides of compounds of the invention by treating with a reducing agent (e.g., sulfur, sulfur dioxide, triphenyl phosphine, lithium borohydride, sodium borohydride, phosphorus trichloride, tribromide, or the like) in an suitable inert organic solvent (e.g., acetonitrile, ethanol, aqueous dioxane, or the like) at 0 to 80° C.

Prodrug derivatives of the compounds of the invention can be prepared by methods known to those of ordinary skill in the art (e.g., for further details see Saulnier et al. (1994), Bioorganic and Medicinal Chemistry Letters, Vol. 4, p. 1985). For example, appropriate prodrugs can be prepared by reacting a non-derivatized compound of the invention with a suitable carbamylating agent (e.g., 1,1-acyloxyalkylcarbonochloridate, para-nitrophenyl carbonate, or the like).

Protected derivatives of the compounds of the invention can be made by means known to those of ordinary skill in the art. A detailed description of the techniques applicable to the creation of protecting groups and their removal can be found in T. W. Greene, Protecting Groups in Organic Synthesis, 3^{rd} edition, John Wiley & Sons, Inc. 1999.

Compounds of the present invention may be conveniently prepared or formed during the process of the invention, as solvates (e.g., hydrates). Hydrates of compounds of the present invention may be conveniently prepared by recrystallisation from an aqueous/organic solvent mixture, using organic solvents such as dioxin, tetrahydrofuran or methanol.

Compounds of the invention can be prepared as their individual stereoisomers by reacting a racemic mixture of the compound with an optically active resolving agent to form a pair of diastereoisomeric compounds, separating the diastereomers and recovering the optically pure enantiomer. While resolution of enantiomers can be carried out using covalent diasteromeric derivatives of compounds of the invention, dissociable complexes are preferred (e.g., crystalline diastereoisomeric salts). Diastereomers have distinct physical properties (e.g., melting points, boiling points, solubilities, reactivity, etc.) and can be readily separated by taking advantage of these dissimilarities. The diastereomers can be separated by chromatography or, preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques Andre Collet, Samuel H. Wilen, Enantiomers, Racemates and Resolutions, John Wiley & Sons, Inc. (1981).

Pharmacology and Utility

The compounds of the invention are selective inhibitors of cysteine proteases, in particular, cathepsin S, K, B, and/or F, and accordingly are useful for treating diseases in which cysteine protease activity contributes to the pathology and/or symptomatology of the disease. For example, the compounds of the invention are useful in treating autoimmune disorders, including, but not limited to, juvenile onset diabetes, psoriasis, multiple sclerosis, pemphigus vulgaris, Graves' disease, myasthenia gravis, systemic lupus erythemotasus, rheumatoid arthritis and Hashimoto's thyroiditis, allergic disorders, including, but not limited to, asthma, allogeneic immune responses, including, but not limited to, organ transplants or tissue grafts and endometriosis.

In particular, the compounds of the invention are inhibitors of Cathepsin B, a lysosomal cysteine protease, and are therefore useful in treating disease states associated with the normal activity or the increased expression of Cathepsin B, for example tumor invasion, metastasis, Alzheimer's Disease, arthritis, inflammatory diseases such as chronic and acute pancreatitis, inflammatory airway disease, and bone and joint disorders, including osteoporosis, osteoarthritis, rheumatoid arthritis, psoriasis, and other autoimmune disorders, liver fibrosis, including liver fibrosis associated with HCV, all types of steatosis (including non-alcoholic steatohepatitis) and alcohol-associated steatohepatitis, non-alcoholic fatty liver disease, forms of pulmonary fibrosis including idiopathic pulmonary fibrosis, pathological diagnosis of interstitial pneumonia following lung biopsy, renal fibrosis, cardiac fibrosis, retinal angiogenesis and fibrosis/gliosis in the eye, schleroderma, and systemic sclerosis. The compounds of the invention may be used alone, or optionally with one or more antiviral agents.

The cysteine protease inhibitory activities of the compounds of the invention can be determined by methods known to those of ordinary skill in the art. Suitable in vitro assays for measuring protease activity and the inhibition thereof by test compounds are known. Typically, the assay measures protease-induced hydrolysis of a peptide-based substrate. Details of assays for measuring protease inhibitory activity are set forth in Biological Examples 6-11, infra.

Administration and Pharmaceutical Compositions

In general, compounds of the invention will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. For example, therapeutically effective amounts of a compound of the invention may range from about 10 micrograms per kilogram body weight (μg/kg) per day to about 20 milligram per kilogram body weight (mg/kg) per day, typically from about 100 μg/kg/day to about 10 mg/kg/day. Therefore, a therapeutically effective amount for an 80 kg human patient may range from about 1 mg/day to about 1.6 g/day, typically from about 1 mg/day to about 100 mg/day. In general, one of ordinary skill in the art, acting in reliance upon personal knowledge and the disclosure of this application, will be able to ascertain a therapeutically effective amount of a compound of the invention for treating a given disease.

The compounds of the invention can be administered as pharmaceutical compositions by one of the following routes: oral, systemic (e.g., transdermal, intranasal or by suppository) or parenteral (e.g., intramuscular, intravenous or subcutaneous). Compositions can take the form of tablets, pills, capsules, semisolids, powders, sustained release formulations, solutions, suspensions, elixirs, aerosols, or any other appropriate composition and are comprised of, in general, a compound of the invention in combination with at least one pharmaceutically acceptable excipient. Acceptable excipients are non-toxic, aid administration, and do not adversely affect the therapeutic benefit of the active ingredient. Such excipient may be any solid, liquid, semisolid or, in the case of an aerosol composition, gaseous excipient that is generally available to one of skill in the art.

### EXAMPLES

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

### Chemical References and Examples

The following illustrates the preparation of intermediates (References) and compounds (Examples) of the invention.

### Reference 1

### 2S-[(4-Fluorophenyl)(trifluoromethyl)methyleneamino]-3-(4-hydroxyphenyl)propanoic acid

A mixture of 1-(4-fluorophenyl)-2,2,2-trifluoroethan-1-one (2 g, 10 mmol, 1 eq), 2-amino-methyl 3S-(4-hydroxyphenyl)propanoate (4 g, 10 mmol, 3 eq) and potassium carbonate (4 g, 10 mmoles, 1 eq) in methanol (20 mL) was stirred at 50° C. for 8 hours. The precipitate was filtered off to afford 2S-[(4-fluorophenyl)(trifluoromethyl)methyleneamino]-3-(4-hydroxyphenyl)propanoic acid (3.5 g, 95%) as an orange powder.

### Reference 2

### 2,2,2-Trifluoro-1S-(4-fluorophenyl)ethylamino-3S-(4-hydroxyphenyl)propanoic acid

A solution of 2S-[(4-fluorophenyl)(trifluoromethyl)methyleneamino]-3-(4-hydroxyphenyl)propanoic acid (3.5 g, 10 mmol, 1 eq), prepared as in Reference 1, in acetonitrile (100 mL) at −40° C. was treated with zinc borohydride (40 mmol, 4 eq) in THF (20 mL). The resulting mixture was stirred at −40° C. for 4 hours and then acetone (20 mL) was added The mixture then was allowed to ware to ambient temperature over 1 hour. Aqueous hydrochloric acid (1N, 50 mL) was added slowly to the mixture. The mixture was condensed and extracted with ethyl acetate (3×100 mL). The organic phase was washed with brine (3×50 mL), dried over Na₂SO₄, filtered and concentrated. The residue was purified by column chromatography (DCM:MeOH=20:1) to give 2,2,2-trifluoro-1S-(4-fluorophenyl)ethylamino-3S-(4-hydroxyphenyl)propanoic acid (2 g, 57%) as a white powder.

### Reference 3

### 2,2,2-Trifluoro-1S-(4-fluorophenyl)ethylamino-3S-(3,5-dichloro-4-hydroxyphenyl)propanoic acid

Sulfuryl chloride (3 g, 9 mmoles, 3 eq) was added dropwise to a solution of 2,2,2-trifluoro-1S-(4-fluorophenyl)ethylamino-3S-(4-hydroxyphenyl)propanoic acid (1 g, 3 mmol, 1 eq), prepared as in Reference 2, in toluene (50 mL). The resulting mixture was stirred at 80° C. for 3 hours and then concentrated. The residue was purified by column chromatography (DCM:MeOH=20:1) to give 2,2,2-trifluoro-1S-(4-fluorophenyl)ethylamino-3S-(3,5-dichloro-4-hydroxyphenyl)propanoic acid (0.6 g, 50%) as a white powder.

### Reference 4

### 2S-[(4-Bromophenyl)(trifluoromethyl)methyleneamino]-3-(4-hydroxyphenyl)propanoic acid

A mixture of 1-(4-bromophenyl)-2,2,2-trifluoroethan-1-one (3 g, 12 mmol, 1 eq), 2-amino-methyl 3S-(4-hydroxyphenyl)propanoate (2.3 g, 12 mmol, 1 eq) and potassium carbonate (4 g, 30 mmoles, 2.5 eq) in methanol (20 mL) was stirred at 50° C. for 8 hours. The precipitate was filtered off to give 2S-[(4-fluorophenyl)(trifluoromethyl)methyleneamino]-3-(4-hydroxyphenyl)propanoic acid (4.2 g, 85%) as an orange powder.

### Reference 5

### 2,2,2-Trifluoro-1S-(4-bromophenyl)ethylamino-3S-(4-hydroxyphenyl)propanoic acid

A solution of 2S-[(4-bromophenyl)(trifluoromethyl)methyleneamino]-3-(4-hydroxyphenyl)propanoic acid (0.5 g, 1 mmol, 1 eq), prepared as in Reference 4, in acetonitrile (100 mL) at −40° C. was treated with zinc borohydride (4 mmol, 4 eq) in THF (10 mL). The resulting mixture was stirred at −40° C. for 4 hours and then acetone (10 mL) was added. The mixture then was allowed to ware to ambient temperature over 1 hour. Aqueous hydrochloric acid (1N, 25 mL) was added slowly to the mixture. The mixture was condensed and extracted with ethyl acetate (3×50 mL). The organic phase was washed with brine (3×50 mL), dried over Na₂SO₄, filtered and concentrated. The residue was purified by column chromatography (DCM:MeOH=20:1) to give 2,2,2-trifluoro-1S-(4-bromophenyl)ethylamino-3S-(4-hydroxyphenyl)propanoic acid (0.3 g, 60%) as a white powder.

### Reference 6

### 2,2,2-Trifluoro-1S-(4-bromophenyl)ethylamino-3S-(3,5-dichloro-4-hydroxyphenyl)propanoic acid

Sulfuryl chloride (0.3 g, 2.1 mmoles, 3 eq) was added dropwise to a solution of 2,2,2-trifluoro-1S-(4-bromophenyl)ethylamino-3S-(4-hydroxyphenyl)propanoic acid (0.3 g, 0.7 mmol, 1 eq), prepared as in Reference 5, in toluene (10 mL). The resulting mixture was stirred at 80° C. for 3 hours and then concentrated. The residue was purified by column chromatography (DCM:MeOH=20:1) to give 2,2,2-trifluoro-1S-(4-bromophenyl)ethylamino-3S-(3,5-dichloro-4-hydroxyphenyl)propanoic acid (0.15 g, 35%) as a white powder.

### Reference 7

### 2,2,2-Trifluoro-1S-(4-bromophenyl)ethylamino-3S-(3,5-diiodo-4-hydroxyphenyl)propanoic acid

A solution of iodine chloride (0.19 g, 1.2 mmoles, 2.4 eq) in acetic acid (1 mL) was added dropwise over 10 minutes to a solution of 2,2,2-trifluoro-1S-(4-bromophenyl)ethylamino-3S-(4-hydroxyphenyl)propanoic acid (0.2 g, 0 5 mmol, 1 eq), prepared as in Reference 5, in acetic acid (5 mL) under nitrogen. The resulting mixture was stirred at ambient temperature for 72 hours and then concentrated. The residue was purified by column chromatography (DCM:MeOH=20:1) to give 2,2,2-trifluoro-1S-(4-bromophenyl)ethylamino-3S-(3,5-diiodo-4-hydroxyphenyl)propanoic acid (0.2 g, 32%) as a white powder.

### Reference 8

### 2,2,2-Trifluoro-1S-(4-fluorophenyl)ethylamino-3S-(3,5-dichloro-4-hydroxyphenyl)propanoic acid

A mixture of methyl (S)-2-amino-3-(3,5-dichloro-4-hydroxy-phenyl)-propionate hydrochloride (992 mg, 3.30 mmol), 1-(4-fluorophenyl)-2,2,2-trifluoroethan-1-one (551 mg, 2.87 mmol) and K₂CO₃ (1.59 g, 11.5 mmol) in methanol (8.3 mL) was stirred for at 50° C. for 18 hours under argon. The mixture was allowed to cool to ambient temperature and diluted with acetonitrile (83 mL). The resulting suspension was transferred within 10 minutes to a cooled (−40° C.) suspension of Zn(BH₄)₂ in DME (prepared from NaBH₄ (1.00 g, 26 4 mmol), ZnCl₂ (1.81 g, 13.2 mmol) and DME (12 mL)). The mixture was stirred at −40° C. for 4 hours and then acetone (10 mL) was added. The reaction was allowed to warm to ambient temperature over 1 hour. The reaction mixture was adjusted to pH 4 with 1M HCl (aprox. 50 mL) and the organic solvents were partially evaporated. The residue was extracted with EtOAc (3×30 mL) and the extract washed with brine and MgSO₄) and concentrated. Purification by column chromatography (eluent CH₂Cl₂/MeOH) and crystallization from CH₂Cl₂ (5 mL) afforded 2,2,2-trifluoro-1S-(4-fluorophenyl)ethylamino-3S-(3,5-dichloro-4-hydroxyphenyl)propanoic acid (0.58 g, 48%).

¹H NMR: δH (400 MHz; DMSO-d6): 10.0-9.8 (1H, br s), 7.56-7.45 (2H, m), 7.32-7.19 (4H, m), 4.60-4.50 (1H, m), 3.43-3.36 (1H, m), 2.91-2.72 (2H, m). ¹⁹F NMR: δF (400 MHz; DMSO-d6): −73.11 (3F, d, J=7.9 Hz), −113.30-(−113.43) (1F, m). dr=28:1

Proceeding as in Reference 8 the following compounds were prepared:

2,2,2-trifluoro-1S-(4-fluorophenyl)ethylamino-3S-(3,5-dichloro-4-hydroxyphenyl)propanoic acid (24%), ¹H NMR: δH (400 MHz; CDCl₃): 7.56-7.49 (2H, m), 7.24-7.19 (2H, m), 7.19-7.15 (2H, m), 4.07-3.98 (1H, m), 3.66-3.60 (1H, m), 3.02-2.85 (2H, m). ¹⁹F NMR: δF (400 MHz; CDCl₃): −73.05 (3F, d, J=7.8 Hz), dr=27:1;

2,2,2-trifluoro-1S-(4-fluorophenyl)ethylamino-3S-(3,5-diiodo-4-hydroxyphenyl)propanoic acid (0.52 g, 37%), ¹H NMR 400 MHz (CDCl₃, ppm) 7.57-7.55 (2H, m), 7.33-7.27 (2H, m), 7.10-7.03 (2H, m), 5.8-5.4 (1H, br s) 4.02 (1H, q, J=7.0 Hz) 3.60 (1H, dd, J=7.0 5.2 Hz), 2.94 (1H, dd, J=14.1 5.2 Hz) 2.83 (1H, dd, J=14.1 7.0 Hz); ¹⁹F NMR: 400 MHz (CDCl₃)-73.93 (3F, d, J=7.3 Hz)-111.53-(−111.64) (1F, m) dr=35:1; and

2,2,2-trifluoro-1S-(4-bromophenyl)ethylamino-3S-(3,5-diiodo-4-hydroxyphenyl)propanoic acid (34%), ¹H NMR: δH (400 MHz; DMSO-d6): 9.5-9.2 (1H, br s), 7.70-7.58 (4H, m), 7.45-7.37 (2H, m), 4.62-4.48 (1H, m), 3.41-3.34 (1H, m), 2.85-2.69 (2H, m). ¹⁹F NMR: δF (400 MHz; DMSO-d6): −72.97 (3F, d, J=7.9 Hz), dr=28:1.

### Reference 9

### (S)-3-amino-N-cyclopropyl-2-oxopentanamide Hydrochloride

Di-tert-butyl dicarbonate (2.4 g, 1.1 mmol) was added to a solution of 3S-amino-N-cyclopropyl-2-hydroxypentanamide hydrochloride (2.1 g, 10 mmol) in THF (100 mL) and saturated NaHCO₃ (50 mL). The mixture was stirred at ambient temperature for 5 hours and then concentrated by evaporation. The residue was partitioned between ethyl acetate and brine. The organic layer was separated, dried (Na₂SO₄), and concentrated. Product was purified from the residue by flash silica gel column chromatography, using 30%-90% gradient of ethyl acetate in hexanes as eluent to yield 3S-tert-butoxycarbonylamino-N-cyclopropyl-2-hydroxypentanamide hydrochloride (2.2 g, 80%). ¹H-NMR 400 MHz (DMSO, ppm). 7.75 (0.6H, d, J=4.2 Hz) and 7.67 (0.4H, d, J=4.6 Hz) 6.33 (0.6H, d, J=9.1 Hz) and 6.00 (0.4H, J=9.4 Hz) 5.48 (0.4H, d, J=5.5 Hz) and 5.31 (0.6H, d, J=6.7 Hz) 3.86-3.77 (1H, m) 3.67-3.54 (1H, m) 2.66-2.54 (1H, m) 1.54-1.18 (2H, m) 1.38 and 1.36 (9H, s) 0.82 and 0.77 (3H, t, J=7.4 Hz) 0.61-0.54 (2H, m) 0.48-0.42 (2H, m).

Dess-Martin reagent (2.5 g, 5.8 mmol) was added to a solution of 3S-tert-butoxycarbonylamino-N-cyclopropyl-2-hydroxypentanamide hydrochloride (1.4 g, 5.1 mmol) in dichloromethane (50 mL). The reaction mixture was stirred at ambient temperature for 3 hours and then extracted with solution of Na₂S₂O₃ (11.8 g, 75 mmol) in saturated NaHCO₃ (100 mL). The organic layer was dried (Na₂SO₄) and concentrated. The product was purified by flash silica gel column chromatography, using 2.5%-10% gradient of ethyl acetate in dichloromethane as eluent to yield title compound 3S-tert-butoxycarbonylamino-N-cyclopropyl-2-oxopentanamide (1.14 g, 83%). ¹H-NMR 400 MHz (DMSO, ppm). 8.71 (1H, d, J=4.9 Hz) 7.20 (1H, d, J=7.5 Hz) 4.70-4.59 (1H, m) 2.78-2.71 (1H, m) 1.78-1.67 (1H, m) 1.51-1.41 (1H, m) 1.36 (9H, s) 0.89 (3H, t, J=7.5 Hz) 0.68-0.62 (2H, m) 0.06-0.54 (2H, m).

6N HCl in isopropanol (15 mL) was added to a solution of 3S-tert-butoxycarbonylamino-N-cyclopropyl-2-oxopentanamide (1.25 g, 4 6 mmol) in dry dichloromethane (30 mL). The reaction mixture was stirred at 40° C. for 40 minutes and then concentrated to give 3S-amino-N-cyclopropyl-2-oxopentanamide hydrochloride (0.95 g, 100%). ¹H-NMR 400 MHz (DMSO, ppm). 9.06 (1H, d, J=5.2 Hz) 8.5-8.2 (1H, br s) 4.74-4.62 (1H, m) 2.84-2.76 (2H, m) 1.98-1.87 (1H, m) 1.85-1.72 (1H, m) 0.91 (3H, t, J=7.5 Hz) 0.73-0.66 (2H, m) 0.64-0.58 (2H, m).

### Example 1

### N-(1-Cyanocyclopropyl)-2S-[2,2,2-trifluoro-1S-(4-fluorophenyl)-ethylamino]-3-(3,5-dichloro-4-hydroxyphenyl)-propanamide

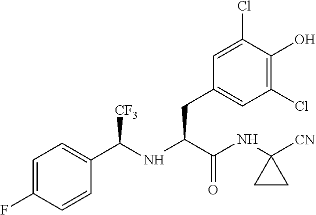 [SMILES: N#CC1(NC(=O)[C@H](Cc2cc(Cl)c(O)c(Cl)c2)N[C@@H](c2ccc(F)cc2)C(F)(F)F)CC1]

A mixture of 2,2,2-trifluoro-1S-(4-fluorophenyl)ethylamino-3S-(3,5-dichloro-4-hydroxyphenyl)propanoic acid (0.4 g, 0.9 mmoles, 1 eq), prepared as in Reference 3, 1-cyanocyclopropylamine (0.11 g, 0.9 mmoles, 1 eq) and diisopropylethylamine (0.9 mmoles, 1 eq) in DMF (15 mL) was stirred at ambient temperature while HATU (0.36 g, 0.9 mmoles, 1 eq) was added. The mixture was stirred for approximately 12 hours at ambient temperature, extracted with ethyl acetate (3×100 mL). The organic layer was washed with brine, dried over sodium sulfate and condensed. The residue was purified by column chromatography (PE:EA=4:1) to give N-(1-cyanocyclopropyl)-2S-[2,2,2-trifluoro-1S-(4-fluorophenyl)-ethylamino]-3-(3,5-dichloro-4-hydroxyphenyl)-propanamide (0.14 g, 30%) as a white powder. ¹NMR (400 MHz, CD₃OD) δ 7.42-7.40 (m, 2H), 7.12-7.09 (m, 4H), 4.20-4.15 (m, 1H), 3.27-3.25 (m, 1H), 2.82 (dd, J=6.8 and 14 Hz, 1H), 2.71 (dd, J=7.6 and 13.6 Hz, 1H), 1.40-1.31 (m, 2H), 0.89-0.85 (m, 1H), 0.76-0.72 (m, 1H).

### Example 2

### N-(1-Cyanocyclopropyl)-2S-[2,2,2-trifluoro-1S-(4-bromophenyl)-ethylamino]-3-(3,5-dichloro-4-hydroxyphenyl)-propanamide

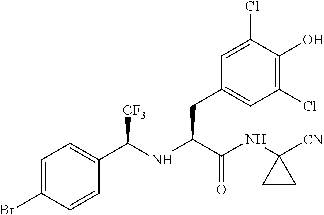 [SMILES: N#CC1(NC(=O)[C@H](Cc2cc(Cl)c(O)c(Cl)c2)N[C@@H](c2ccc(Br)cc2)C(F)(F)F)CC1]

A mixture of 2,2,2-trifluoro-1S-(4-bromophenyl)ethylamino-3 S-(3,5-dichloro-4-hydroxyphenyl)propanoic acid (0.3 g, 0.6 mmoles, 1 eq), prepared as in Reference 6, 1-cyanocyclopropylamine (0.075 g, 0.6 mmoles, 1 eq) and diisopropylethylamine (0.6 mmoles, 1 eq) in DMF (15 mL) was stirred at ambient temperature while HATU (0.23 g, 0.6 mmoles, 1 eq) was added. The mixture was stirred for approximately 12 hours at ambient temperature, extracted with ethyl acetate (3×100 mL). The organic layer was washed with brine (50 mL) and condensed. The residue was purified by column chromatography (PE:EA=4:1) to give N-(1-cyanocyclopropyl)-2S-[2,2,2-trifluoro-1S-(4-bromophenyl)-ethylamino]-3-(3,5-dichloro-4-hydroxyphenyl)-propanamide (0.60 mg, 18%) as a white powder. LC-MS: 550.15 (m-H)⁻. ¹NMR (400 MHz, CD₃OD) δ 7.52 (d, J=8.0 Hz, 2H), 7.29 (d, J=8.0 Hz, 2H), 7.09 (s, 2H), 4.18-4.15 (m, 1H), 3.27-3.25 (m, 1H), 2.82 (dd, J=6.8 and 13.6 Hz, 1H), 2.71 (dd, J=8.0 and 14.0 Hz, 1H), 1.39-1.27 (m, 2H), 0.88-0.85 (m, 1H), 0.73-0.71 (m, 1H).

### Example 3

### N-Cyanomethyl-2S-[2,2,2-trifluoro-1S-(4-bromophenyl)-ethylamino]-3-(3,5-diiodo-4-hydroxyphenyl)-propanamide

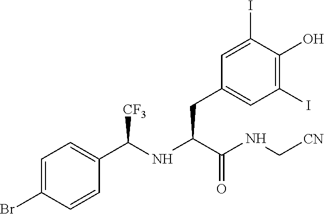 [SMILES: N#CCNC(=O)[C@H](Cc1cc(I)c(O)c(I)c1)N[C@@H](c1ccc(Br)cc1)C(F)(F)F]

A mixture of 2,2,2-trifluoro-1S-(4-bromophenyl)ethylamino-3S-(3,5-diiodo-4-hydroxyphenyl)propanoic acid (0.4 g, 0.6 mmoles, 1 eq), prepared as in Reference 7, cyanomethylamine (0.056 g, 0.6 mmoles, 1 eq) and diisopropylethylamine (0.6 mmoles, 1 eq) in DMF (15 mL) was stirred at ambient temperature while HATU (0.23 g, 0.6 mmoles, 1 eq) was added. The mixture was stirred for approximately 12 hours at ambient temperature, extracted with ethyl acetate (3×100 mL). The organic layer was washed with brine (50 mL) and condensed by vacuum. The residue was purified by column chromatography (PE:EA=4:1) to give N-cyanomethyl-2S-[2,2,2-trifluoro-1S-(4-bromophenyl)-ethylamino]-3-(3,5-diiodo-4-hydroxyphenyl)-propanamide (0.15 g, 36%) as a white powder. LC-MS: 706.30 (m-H)⁻. ¹NMR (400 MHz, DMSO-d6) δ 8.64 (br s, 1H), 7.56-7.54 (m, 4H), 7.32 (d, J=7.6 Hz, 2H), 4.30-4.28 (m, 1H), 4.01-3.95 (m, 3H), 3.15-3.13 (m, 1H), 3.02-3.00 (m, 2H); m/z.

### Example 4

### N-(1-Cyanocyclopropyl)-2S-[2,2,2-trifluoro-1S-(4-bromophenyl)-ethylamino]-3-(3,5-diiodo-4-hydroxyphenyl)-propanamide

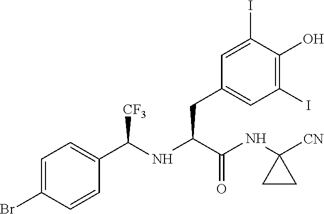 [SMILES: N#CC1(NC(=O)[C@H](Cc2cc(I)c(O)c(I)c2)N[C@@H](c2ccc(Br)cc2)C(F)(F)F)CC1]

A mixture of 2,2,2-trifluoro-1S-(4-bromophenyl)ethylamino-3S-(3,5-dichloro-4-hydroxyphenyl)propanoic acid (0.4 g, 0.6 mmoles, 1 eq), prepared as in Reference 7, 1-cyanocyclopropylamine (0.075 g, 0.6 mmoles, 1 eq) and diisopropylethylamine (0.6 mmoles, 1 eq) in DMF (10 mL) was stirred at ambient temperature while HATU (0.23 g, 0.6 mmoles, 1 eq) was added. The mixture was stirred for approximately 12 hours at ambient temperature, extracted with ethyl acetate (3×100 mL). The organic layer was washed with brine (50 mL) and condensed by vacuum. The residue was purified by column chromatography (PE:EA=4:1) to N-(1-cyanocyclopropyl)-2S-[2,2,2-trifluoro-1S-(4-bromophenyl)-ethylamino]-3-(3,5-diiodo-4-hydroxyphenyl)-propanamide (0.1 g, 23%) as a white powder. LC-MS: 734.30 (M-H)⁻. 1H¹NMR (400 MHz, CD₃OD) δ 7.54-7.52 (m, 4H), 7.32-7.30 (m, 2H), 4.17-4.14 (m, 1H), 3.26-3.24 (m, 1H), 2.80-2.69 (m, 2H), 1.36-1.27 (m, 2H), 0.88-0.72 (m, 2H), m/z.

### Example 5

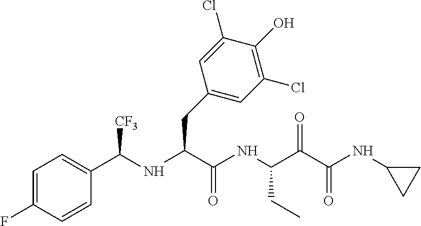 [SMILES: CC[C@H](NC(=O)[C@H](Cc1cc(Cl)c(O)c(Cl)c1)N[C@@H](c1ccc(F)cc1)C(F)(F)F)C(=O)C(=O)NC1CC1]

### N-Cyclopropyl-3S-{3-[3,5-dichloro-4-hydroxyphenyl)-2S-(2,2,2-trifluoro)-1S-(4-fluorophenyl)ethylamino]propionylamino}-2-oxopentanamide

A solution of N-methylmorpholine (102 μl, 0.94 mmol) in dry dichloromethane (2 mL) was added dropwise with a syringe pump over 1 hour to a mixture of (S)-3-(3,5-dichloro-4-hydroxy-phenyl)-2-[(S)-2,2,2-trifluoro-1-(4-fluoro-phenyl)-ethylamino]-propionic acid (100 mg, 0.23 mmol), prepared as in Reference 8, (S)-3-amino-N-cyclopropyl-2-oxopentanamide hydrochloride (95 mg, 0.46 mmol), prepared as in Reference 8, and HATU (98 mg, 0.25 mmol) in dry dichloromethane (3 mL) at ambient temperature. The reaction mixture was stirred for 20 minutes and then ethyl acetate (20 mL) and 10% citric acid (10 mL) were added. The organic layer was separated and extracted with saturated NaHCO₃ (10 mL) and brine (10 mL). The extract was dried (Na₂SO₄) and concentrated by evaporation. Purification by column chromatography (eluent EtOAc/hexanes) and crystallization from Et₂O/hexanes (5 mL) afforded N-cyclopropyl-3S-{3-[3,5-dichloro-4-hydroxyphenyl)-2S-(2,2,2-trifluoro)-1S-(4-fluorophenyl)ethylamino]propionylamino}-2-oxopentanamide (48 mg, 36%).

¹H-NMR 400 MHz (DMSO, ppm) δ: 10.0-9.7 (1H, br s) 8.70 (1H, d, J=5.2 Hz) 8.26 (1H, d, J=7.1 Hz) 7.46-7.39 (2H, m) 7.24 (2H, s) 7.23-7.17 (2H, m) 4.80-4.72 (1H, m) 4.30-4.21 (1H, m) 3.50-3.41 (1H, m) 2.94 (1H, dd, J=10.0 5.8 Hz) 2.78-2.59 (3H, m) 1.75-1.63 (1H, m) 1.50-1.37 (1H, m) 0.77 (3H, t, J=7.5 Hz) 0.68-0.54 (4H, m). ¹⁹F-NMR 400 MHz (DMSO, ppm) δ: −72.87 (3F, d, J=8.2 Hz)-113.31-(−113.44) (1F, m).

Proceeding as in Example 8 the following compounds of the invention were prepared:

N-cyclopropyl-3S-{3-[3,5-dichloro-4-hydroxyphenyl)-2S-(2,2,2-trifluoro)-1S-(4-bromophenyl)ethylamino]propionylamino}-2-oxopentanamide (40%), ¹H-NMR 400 MHz (DMSO, ppm) 9.9-9.8 (1H, br s) δ: 8.70 (1H, d, J=5.3 Hz) 8.69 (1H, d, J=7.0 Hz) 7.60-7.54 (2H, m) 7.37-7.31 (2H, m) 7.24 (2H, s) 4.78-4.71 (1H, m) 4.30-4.20 (1H, m) 3.48-3.40 (1H, m) 2.97 (1H, dd, J=9.8 6.0 Hz) 2.77-2.58 (3H, m) 1.74-1.62 (1H, m) 1.48-1.37 (1H, m) 0.76 (3H, t, J=7.5 Hz) 0.68-0.54 (4H, m). ¹⁹F-NMR 400 MHz (DMSO, ppm) δ: −72.81 (3F, d, J=8.2 Hz);

N-cyclopropyl-3S-{3-[3,5-diiodo-4-hydroxyphenyl)-2S-(2,2,2-trifluoro)-1S-(4-fluorophenyl)ethylamino]propionylamino}-2-oxopentanamide (24%), ¹H-NMR 400 MHz (DMSO-d₆, ppm) 9.4-9.2 (1H, br s) 8.69 (1H, d, J=5.1 Hz) 8.26 (1H, d, J=6.9 Hz) 7.6 4 (2H, s) 7.46-7.38 (2H, m) 7.25-7.16 (2H, m) 4.80-4.18 (1H, m) 4.29-4.18 (1H, m) 3.46-3.36 (1H, m) 2.92 (1H, dd, J=9.9 5.8 Hz) 2.79-2.57 (3H, m) 1.75-1.63 (1H, m) 1.50-1.38 (1H, m) 0.78 (3H, t, J=7.4 Hz) 0.70-0.53 (4H, m). ¹⁹F-NMR 400 MHz (DMSO-d₆, ppm)-72.78 (3F, d, J=7.5 Hz)-113.24−(−113.43) (1F, m), dr=27:1; and

N-cyclopropyl-3S-{3-[3,5-diiodo-4-hydroxyphenyl)-2S-(2,2,2-trifluoro)-1S-(4-bromophenyl)ethylamino]propionylamino}-2-oxopentanamide (49%), ¹H-NMR 400 MHz (DMSO-d₆, ppm) 9.4-9.2 (1H, br s) 8.69 (1H, d, J=5.1 Hz) 8.24 (1H, d, J=7.1 Hz) 7.64 (2H, s) 7.59-7.54 (2H, m) 7.36-7.30 (2H, m) 4.77-4.69 (1H, m) 4.29-4.18 (1H, m) 3.44-3.36 (1H, m) 2.96 (1H, dd, J=9.8 6.0 Hz) 2.76-2.70 (1H, m) 2.69-2.54 (2H, m) 1.71-1.63 (1H, m).

Proceeding as in the References and Examples above the following compounds of the Invention are prepared.

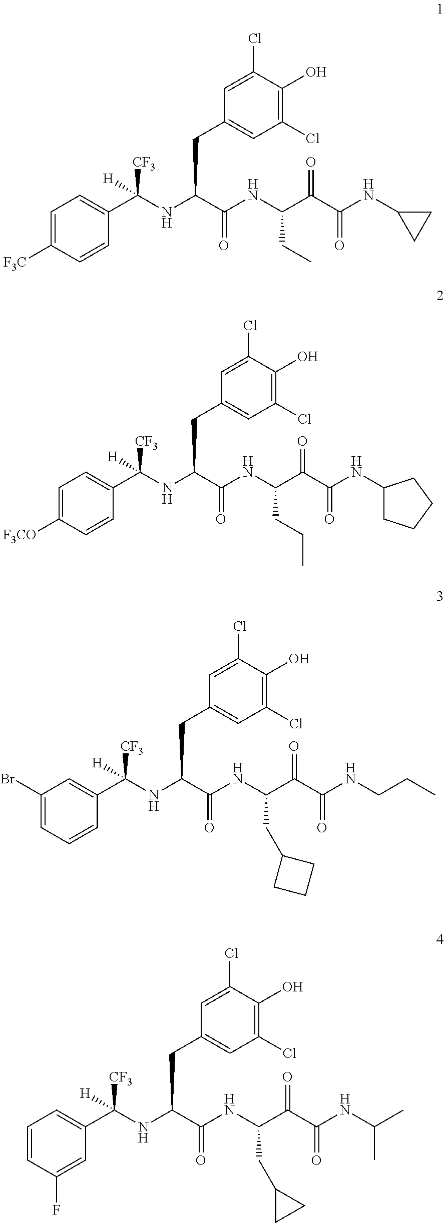 [SMILES: CC[C@H](NC(=O)[C@H](Cc1cc(Cl)c(O)c(Cl)c1)N[C@@H](c1ccc(C(F)(F)F)cc1)C(F)(F)F)C(=O)C(=O)NC1CC1 ; CCC[C@H](NC(=O)[C@H](Cc1cc(Cl)c(O)c(Cl)c1)N[C@@H](c1ccc(OC(F)(F)F)cc1)C(F)(F)F)C(=O)C(=O)NC1CCCC1 ; CCCNC(=O)C(=O)[C@H](CC1CCC1)NC(=O)[C@H](Cc1cc(Cl)c(O)c(Cl)c1)N[C@@H](c1cccc(Br)c1)C(F)(F)F ; CC(C)NC(=O)C(=O)[C@H](CC1CC1)NC(=O)[C@H](Cc1cc(Cl)c(O)c(Cl)c1)N[C@@H](c1cccc(F)c1)C(F)(F)F]

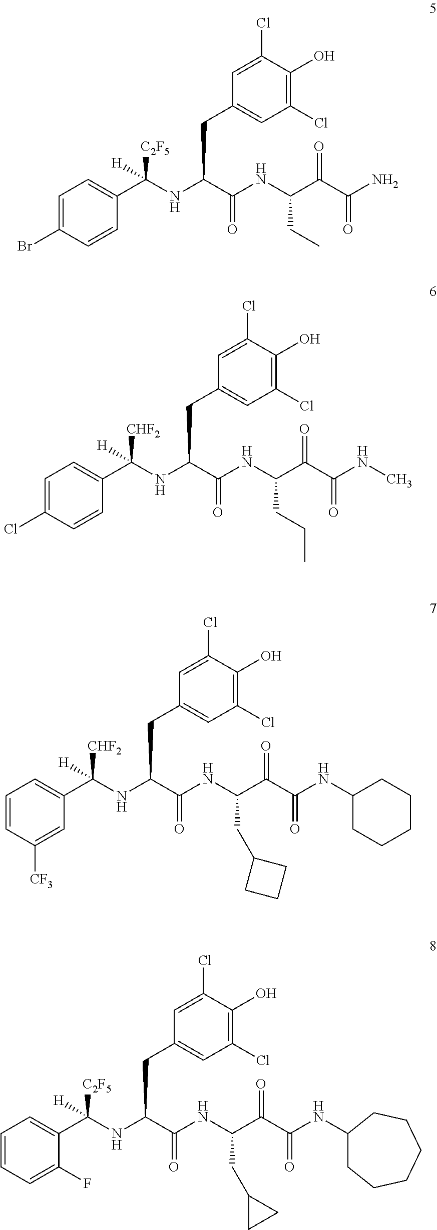

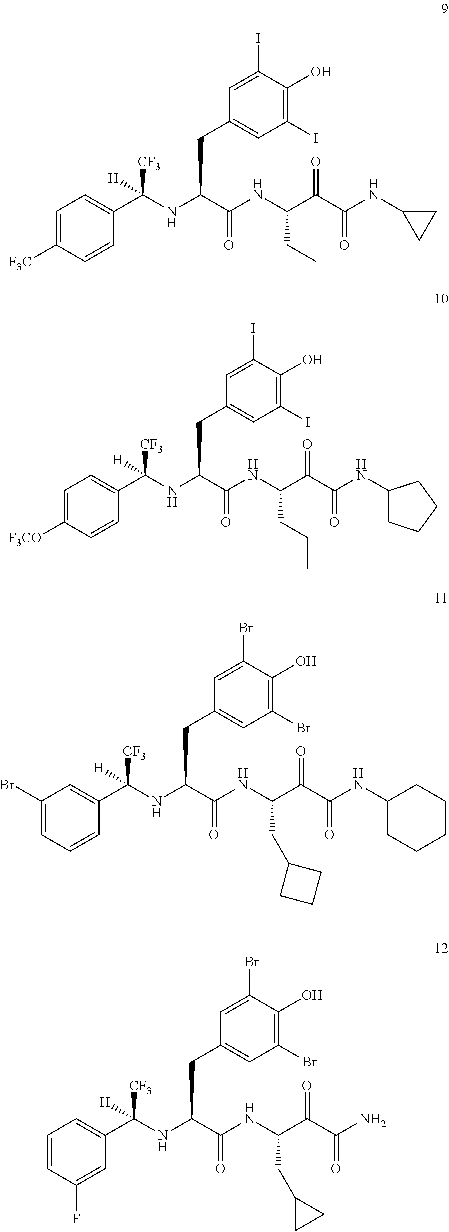 [SMILES: CC[C@H](NC(=O)[C@H](Cc1cc(I)c(O)c(I)c1)N[C@@H](c1ccc(C(F)(F)F)cc1)C(F)(F)F)C(=O)C(=O)NC1CC1 ; CCC[C@H](NC(=O)[C@H](Cc1cc(I)c(O)c(I)c1)N[C@@H](c1ccc(OC(F)(F)F)cc1)C(F)(F)F)C(=O)C(=O)NC1CCCC1 ; O=C(NC1CCCCC1)C(=O)[C@H](CC1CCC1)NC(=O)[C@H](Cc1cc(Br)c(O)c(Br)c1)N[C@@H](c1cccc(Br)c1)C(F)(F)F ; NC(=O)C(=O)[C@H](CC1CC1)NC(=O)[C@H](Cc1cc(Br)c(O)c(Br)c1)N[C@@H](c1cccc(F)c1)C(F)(F)F]

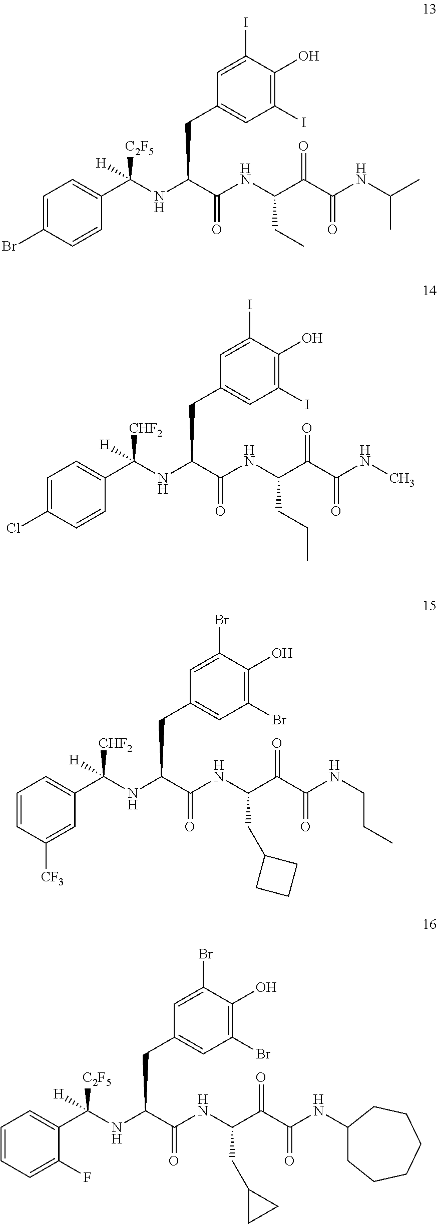

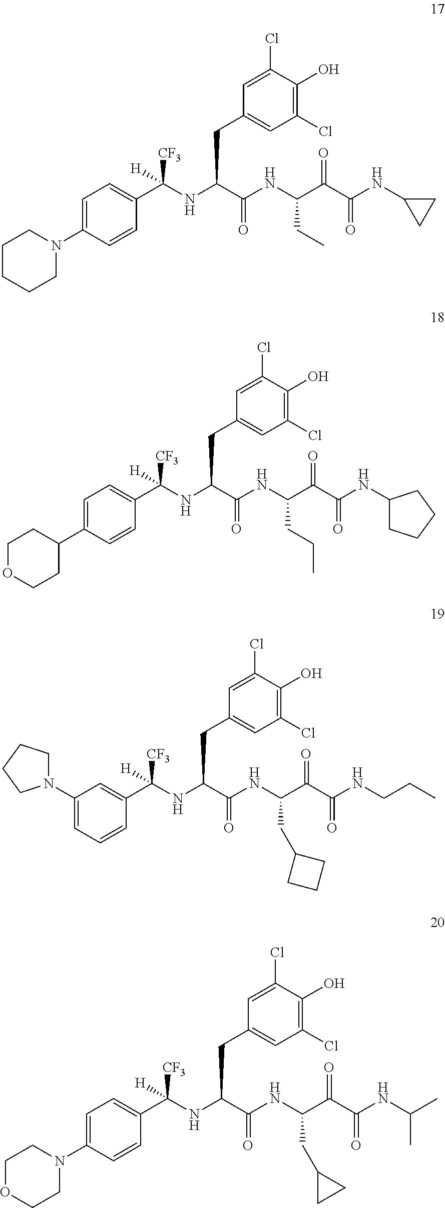 [SMILES: CC[C@H](NC(=O)[C@H](Cc1cc(Cl)c(O)c(Cl)c1)N[C@@H](c1ccc(N2CCCCC2)cc1)C(F)(F)F)C(=O)C(=O)NC1CC1 ; CCC[C@H](NC(=O)[C@H](Cc1cc(Cl)c(O)c(Cl)c1)N[C@@H](c1ccc(C2CCOCC2)cc1)C(F)(F)F)C(=O)C(=O)NC1CCCC1 ; CCCNC(=O)C(=O)[C@H](CC1CCC1)NC(=O)[C@H](Cc1cc(Cl)c(O)c(Cl)c1)N[C@@H](c1cccc(N2CCCC2)c1)C(F)(F)F ; CC(C)NC(=O)C(=O)[C@H](CC1CC1)NC(=O)[C@H](Cc1cc(Cl)c(O)c(Cl)c1)N[C@@H](c1ccc(N2CCOCC2)cc1)C(F)(F)F]

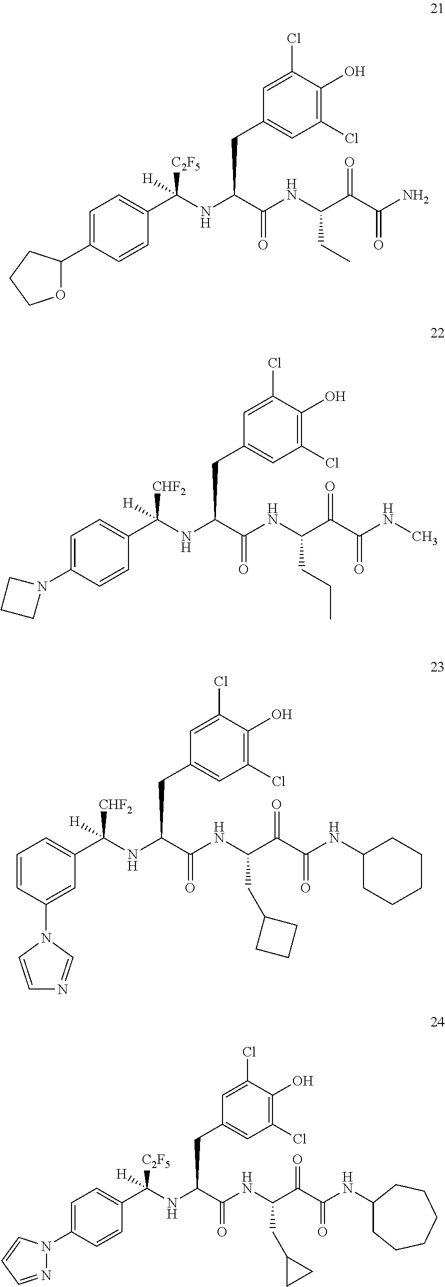

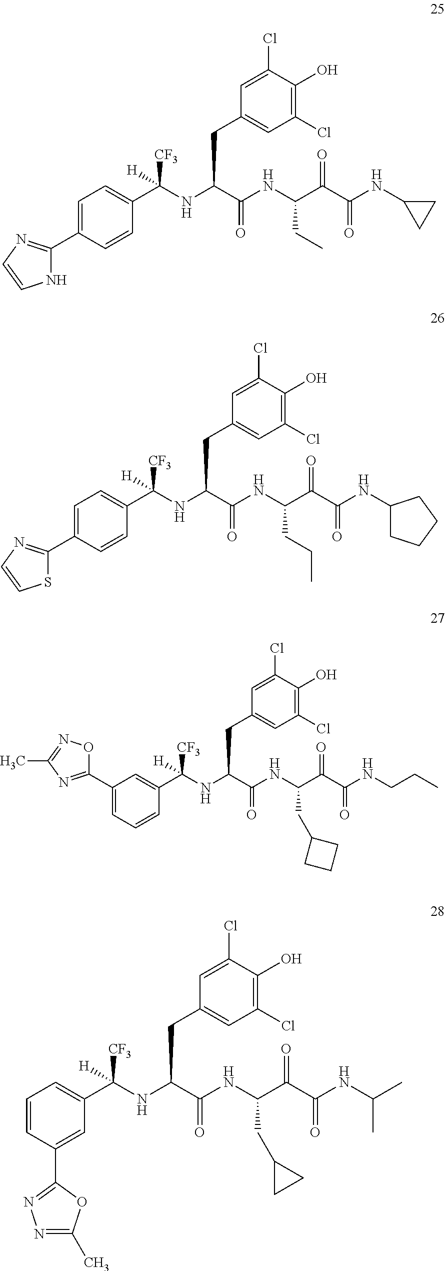 [SMILES: CC[C@H](NC(=O)[C@H](Cc1cc(Cl)c(O)c(Cl)c1)N[C@@H](c1ccc(-c2ncc[nH]2)cc1)C(F)(F)F)C(=O)C(=O)NC1CC1 ; CCC[C@H](NC(=O)[C@H](Cc1cc(Cl)c(O)c(Cl)c1)N[C@@H](c1ccc(-c2nccs2)cc1)C(F)(F)F)C(=O)C(=O)NC1CCCC1 ; CCCNC(=O)C(=O)[C@H](CC1CCC1)NC(=O)[C@H](Cc1cc(Cl)c(O)c(Cl)c1)N[C@@H](c1cccc(-c2nc(C)no2)c1)C(F)(F)F ; Cc1nnc(-c2cccc([C@H](N[C@@H](Cc3cc(Cl)c(O)c(Cl)c3)C(=O)N[C@@H](CC3CC3)C(=O)C(=O)NC(C)C)C(F)(F)F)c2)o1]

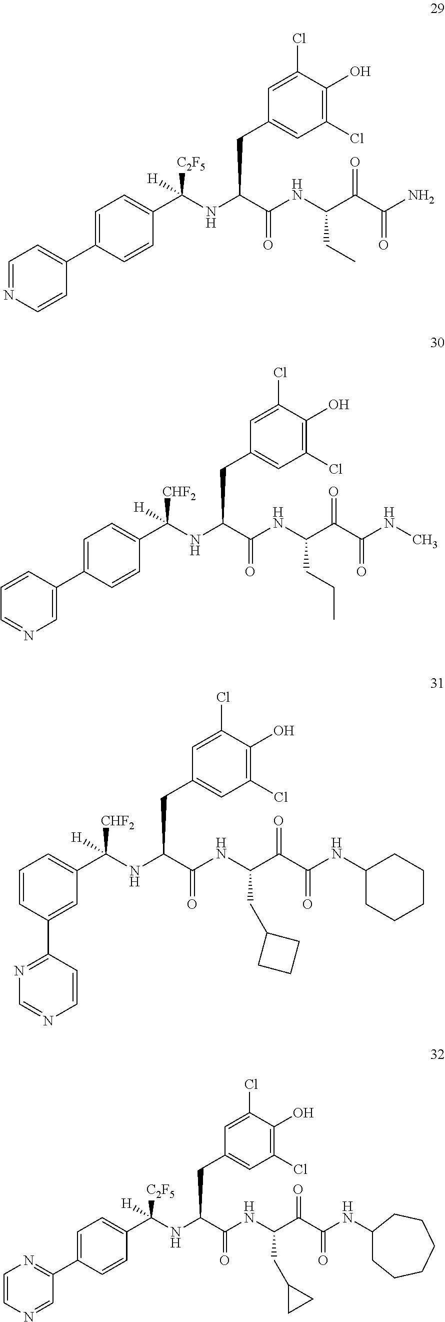

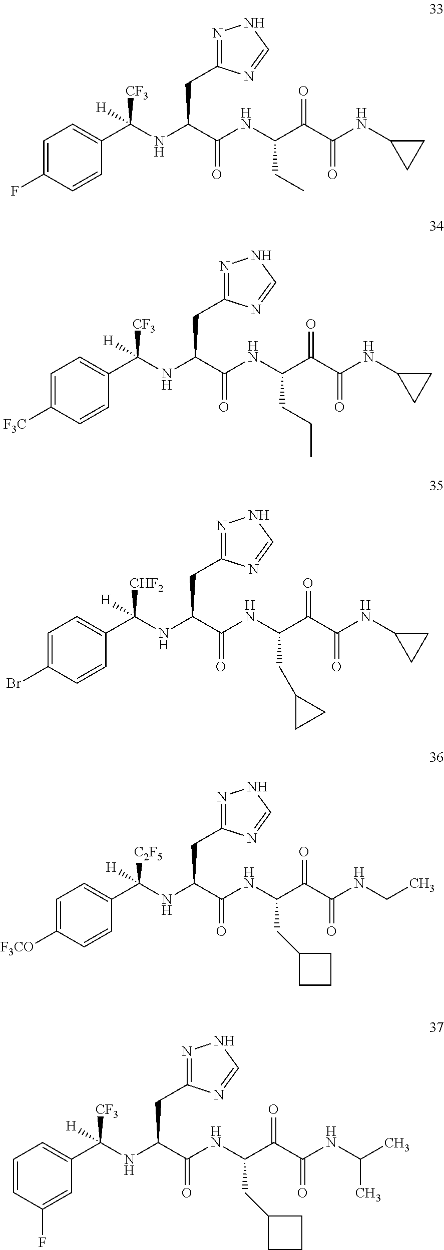

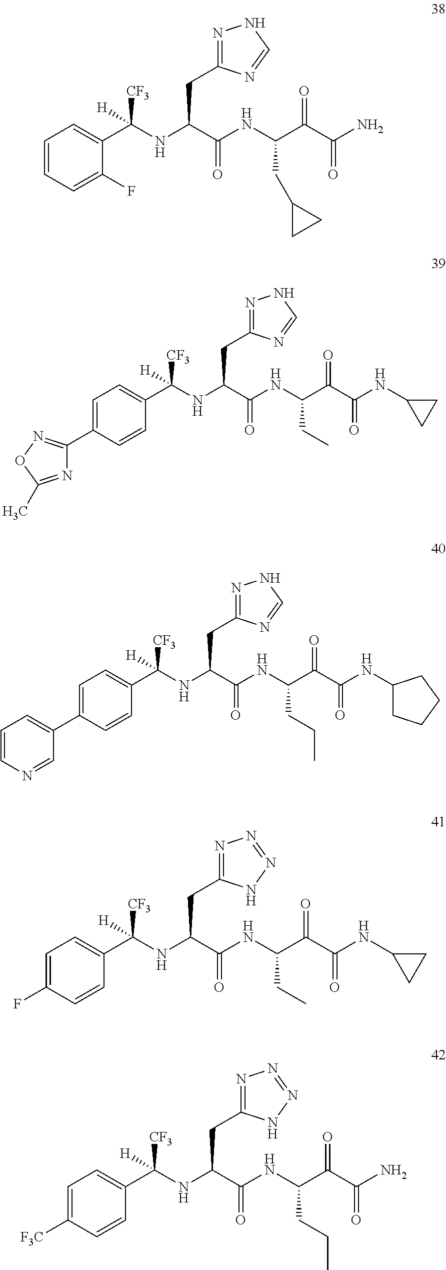 [SMILES: NC(=O)C(=O)[C@H](CC1CC1)NC(=O)[C@H](Cc1nc[nH]n1)N[C@@H](c1ccccc1F)C(F)(F)F ; CC[C@H](NC(=O)[C@H](Cc1nc[nH]n1)N[C@@H](c1ccc(-c2noc(C)n2)cc1)C(F)(F)F)C(=O)C(=O)NC1CC1 ; CCC[C@H](NC(=O)[C@H](Cc1nc[nH]n1)N[C@@H](c1ccc(-c2cccnc2)cc1)C(F)(F)F)C(=O)C(=O)NC1CCCC1 ; CC[C@H](NC(=O)[C@H](Cc1nnn[nH]1)N[C@@H](c1ccc(F)cc1)C(F)(F)F)C(=O)C(=O)NC1CC1 ; CCC[C@H](NC(=O)[C@H](Cc1nnn[nH]1)N[C@@H](c1ccc(C(F)(F)F)cc1)C(F)(F)F)C(=O)C(N)=O]

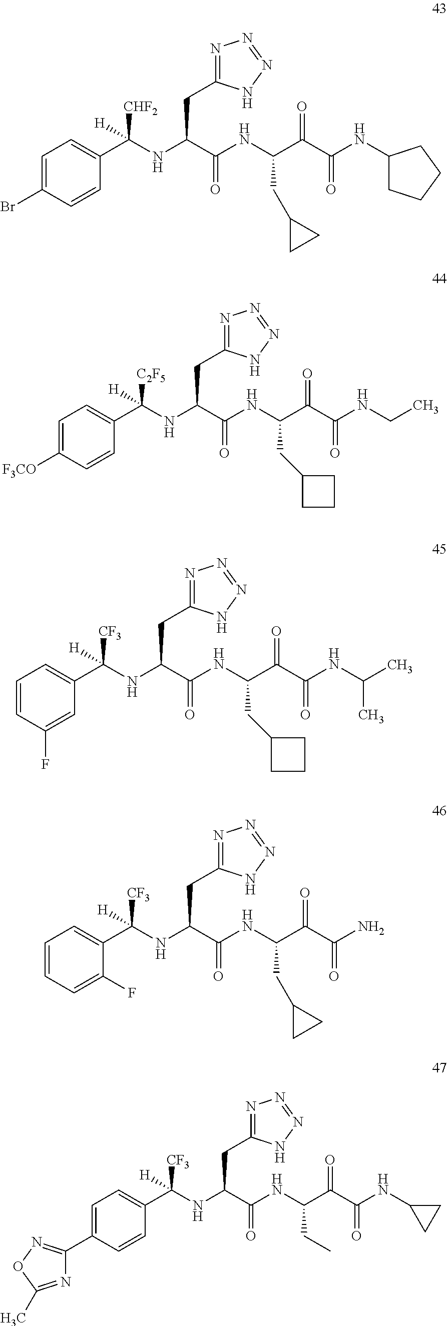

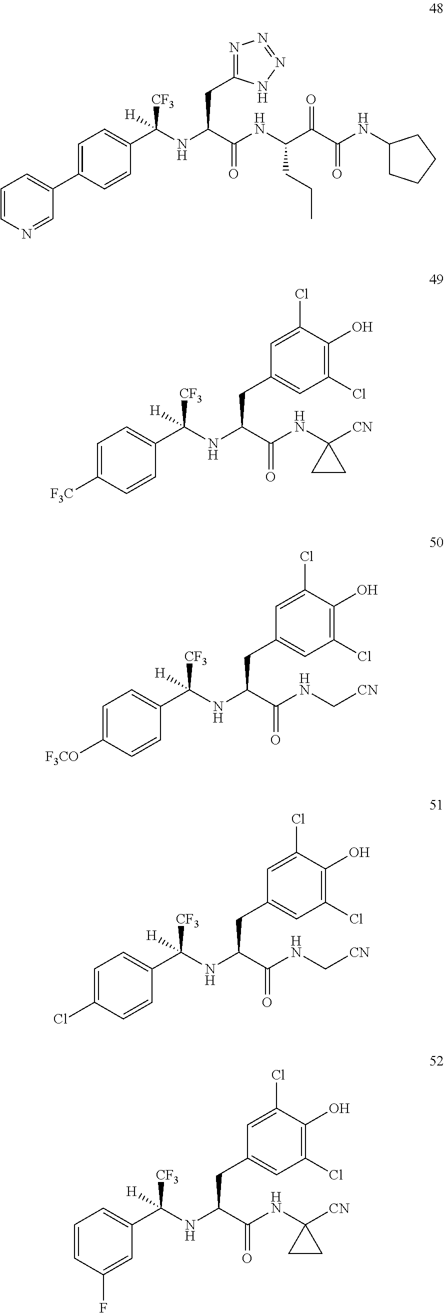 [SMILES: CCC[C@H](NC(=O)[C@H](Cc1nnn[nH]1)N[C@@H](c1ccc(-c2cccnc2)cc1)C(F)(F)F)C(=O)C(=O)NC1CCCC1 ; N#CC1(NC(=O)[C@H](Cc2cc(Cl)c(O)c(Cl)c2)N[C@@H](c2ccc(C(F)(F)F)cc2)C(F)(F)F)CC1 ; N#CCNC(=O)[C@H](Cc1cc(Cl)c(O)c(Cl)c1)N[C@@H](c1ccc(OC(F)(F)F)cc1)C(F)(F)F ; N#CCNC(=O)[C@H](Cc1cc(Cl)c(O)c(Cl)c1)N[C@@H](c1ccc(Cl)cc1)C(F)(F)F ; N#CC1(NC(=O)[C@H](Cc2cc(Cl)c(O)c(Cl)c2)N[C@@H](c2cccc(F)c2)C(F)(F)F)CC1]

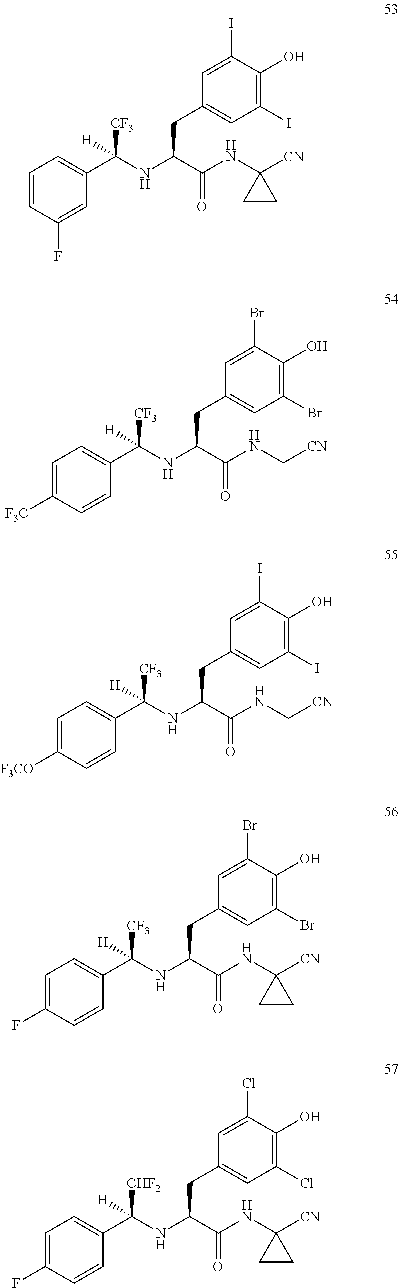 [SMILES: N#CC1(NC(=O)[C@H](Cc2cc(I)c(O)c(I)c2)N[C@@H](c2cccc(F)c2)C(F)(F)F)CC1 ; N#CCNC(=O)[C@H](Cc1cc(Br)c(O)c(Br)c1)N[C@@H](c1ccc(C(F)(F)F)cc1)C(F)(F)F ; N#CCNC(=O)[C@H](Cc1cc(I)c(O)c(I)c1)N[C@@H](c1ccc(OC(F)(F)F)cc1)C(F)(F)F ; N#CC1(NC(=O)[C@H](Cc2cc(Br)c(O)c(Br)c2)N[C@@H](c2ccc(F)cc2)C(F)(F)F)CC1 ; N#CC1(NC(=O)[C@H](Cc2cc(Cl)c(O)c(Cl)c2)N[C@@H](c2ccc(F)cc2)C(F)F)CC1]

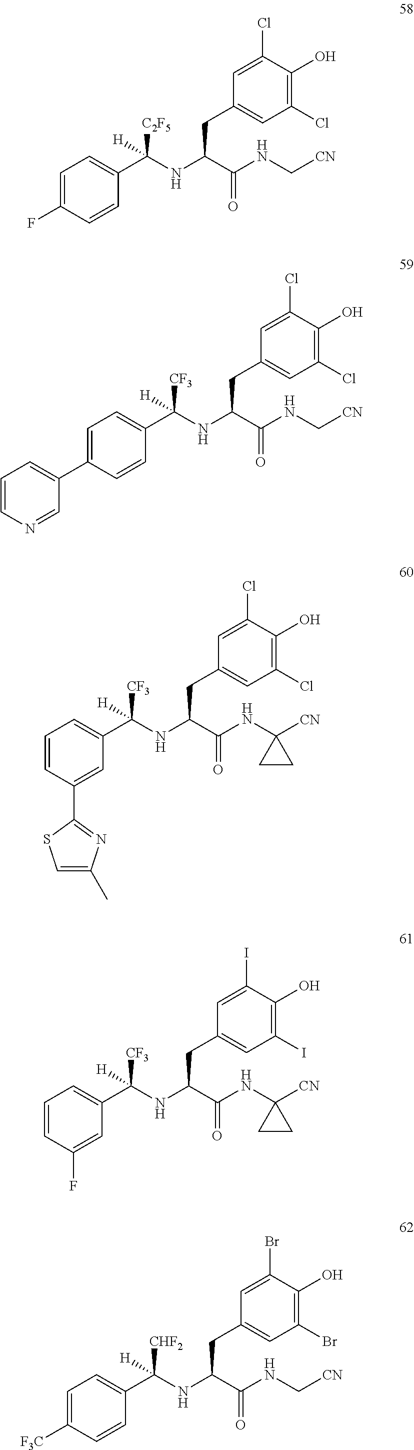

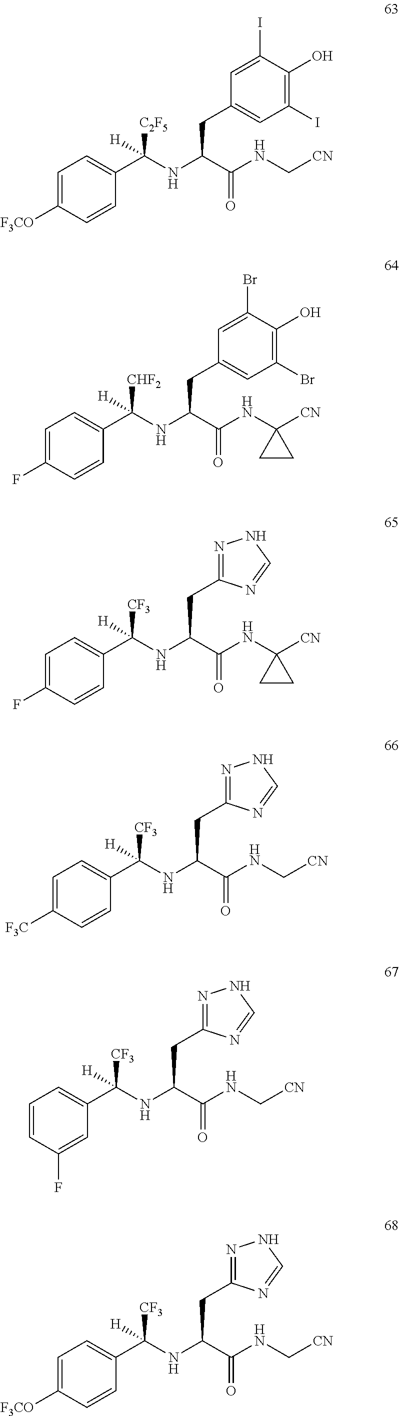

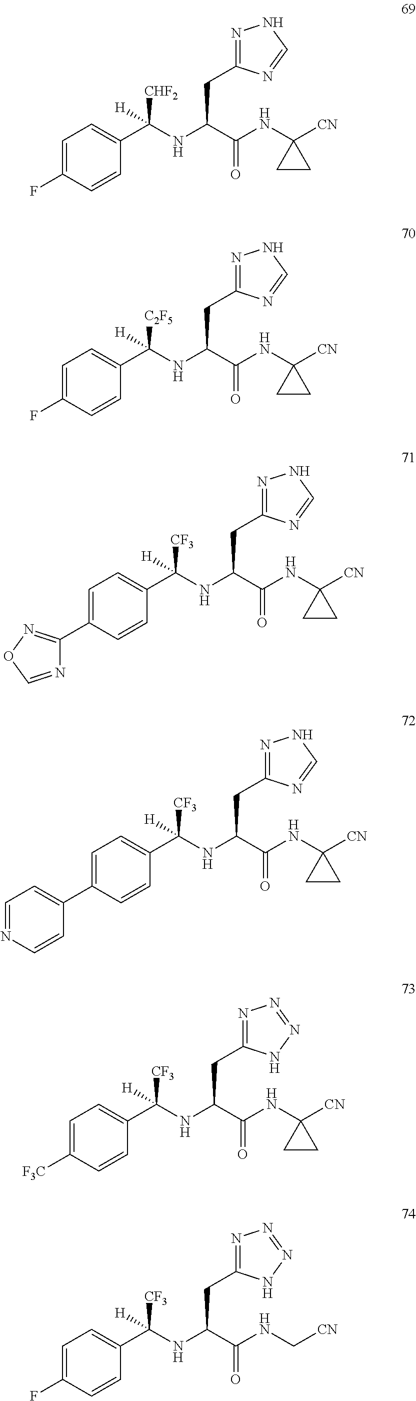

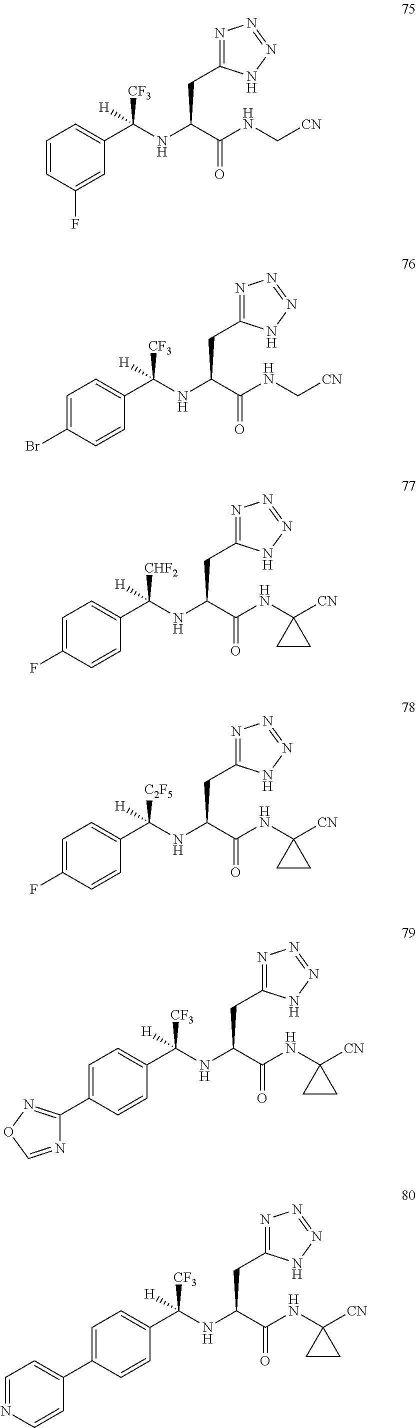

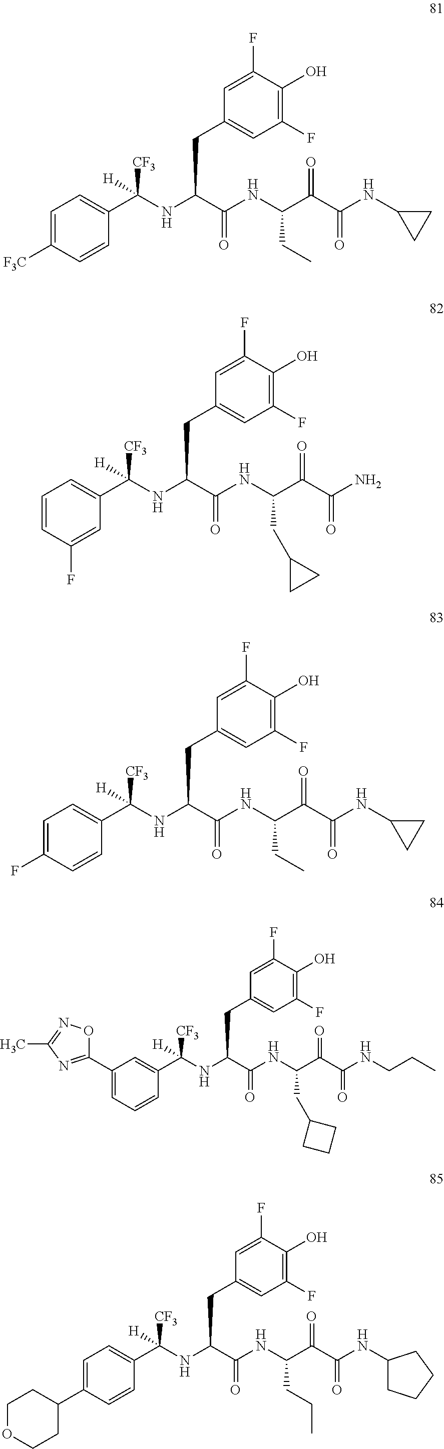 [SMILES: CC[C@H](NC(=O)[C@H](Cc1cc(F)c(O)c(F)c1)N[C@@H](c1ccc(C(F)(F)F)cc1)C(F)(F)F)C(=O)C(=O)NC1CC1 ; NC(=O)C(=O)[C@H](CC1CC1)NC(=O)[C@H](Cc1cc(F)c(O)c(F)c1)N[C@@H](c1cccc(F)c1)C(F)(F)F ; CC[C@H](NC(=O)[C@H](Cc1cc(F)c(O)c(F)c1)N[C@@H](c1ccc(F)cc1)C(F)(F)F)C(=O)C(=O)NC1CC1 ; CCCNC(=O)C(=O)[C@H](CC1CCC1)NC(=O)[C@H](Cc1cc(F)c(O)c(F)c1)N[C@@H](c1cccc(-c2nc(C)no2)c1)C(F)(F)F ; CCC[C@H](NC(=O)[C@H](Cc1cc(F)c(O)c(F)c1)N[C@@H](c1ccc(C2CCOCC2)cc1)C(F)(F)F)C(=O)C(=O)NC1CCCC1]

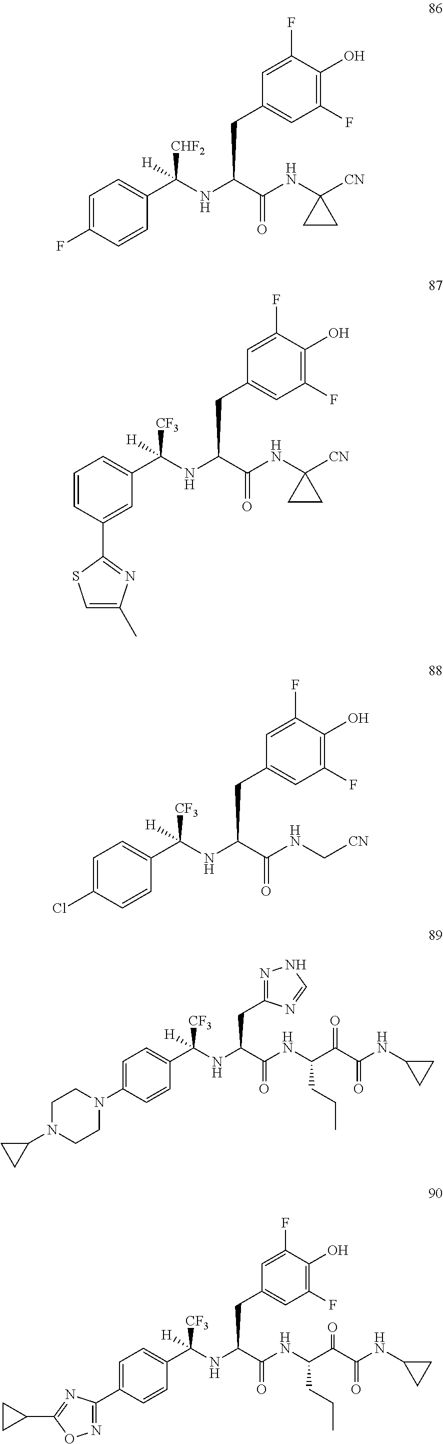 [SMILES: N#CC1(NC(=O)[C@H](Cc2cc(F)c(O)c(F)c2)N[C@@H](c2ccc(F)cc2)C(F)F)CC1 ; Cc1csc(-c2cccc([C@H](N[C@@H](Cc3cc(F)c(O)c(F)c3)C(=O)NC3(C#N)CC3)C(F)(F)F)c2)n1 ; N#CCNC(=O)[C@H](Cc1cc(F)c(O)c(F)c1)N[C@@H](c1ccc(Cl)cc1)C(F)(F)F ; CCC[C@H](NC(=O)[C@H](Cc1nc[nH]n1)N[C@@H](c1ccc(N2CCN(C3CC3)CC2)cc1)C(F)(F)F)C(=O)C(=O)NC1CC1 ; CCC[C@H](NC(=O)[C@H](Cc1cc(F)c(O)c(F)c1)N[C@@H](c1ccc(-c2noc(C3CC3)n2)cc1)C(F)(F)F)C(=O)C(=O)NC1CC1]

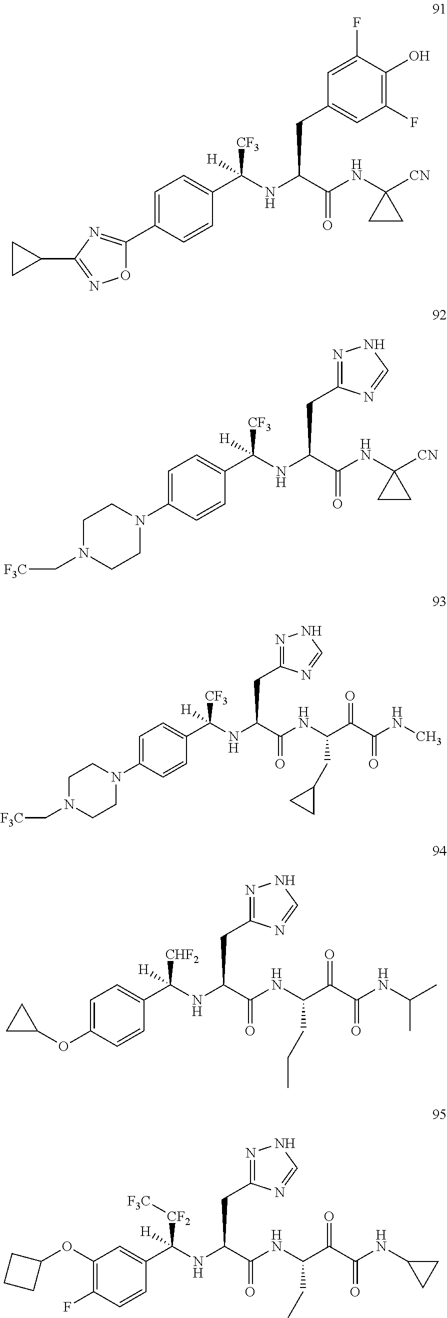 [SMILES: N#CC1(NC(=O)[C@H](Cc2cc(F)c(O)c(F)c2)N[C@@H](c2ccc(-c3nc(C4CC4)no3)cc2)C(F)(F)F)CC1 ; N#CC1(NC(=O)[C@H](Cc2nc[nH]n2)N[C@@H](c2ccc(N3CCN(CC(F)(F)F)CC3)cc2)C(F)(F)F)CC1 ; CNC(=O)C(=O)[C@H](CC1CC1)NC(=O)[C@H](Cc1nc[nH]n1)N[C@@H](c1ccc(N2CCN(CC(F)(F)F)CC2)cc1)C(F)(F)F ; CCC[C@H](NC(=O)[C@H](Cc1nc[nH]n1)N[C@@H](c1ccc(OC2CC2)cc1)C(F)F)C(=O)C(=O)NC(C)C ; CC[C@H](NC(=O)[C@H](Cc1nc[nH]n1)N[C@@H](c1ccc(F)c(OC2CCC2)c1)C(F)(F)C(F)(F)F)C(=O)C(=O)NC1CC1]

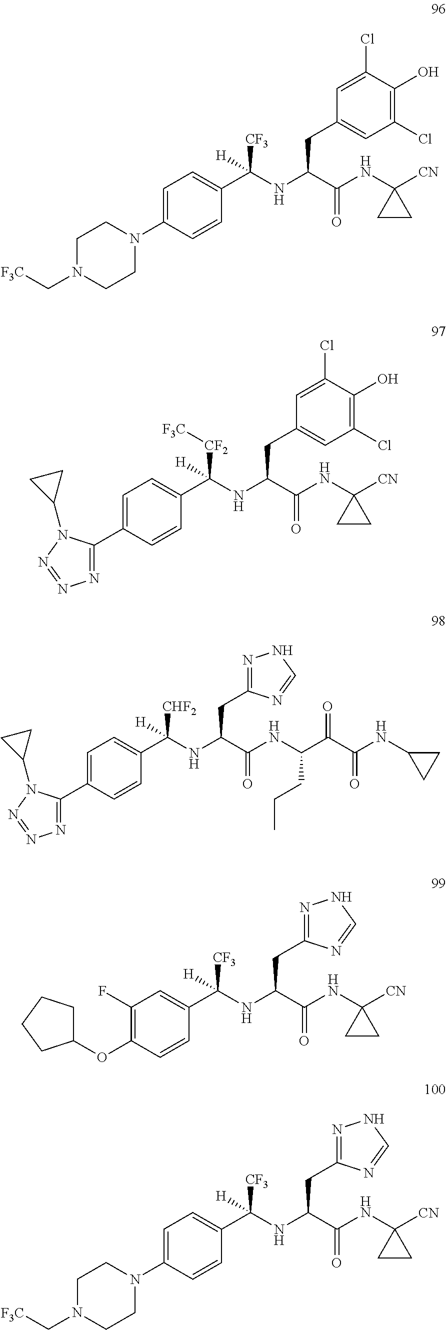 [SMILES: N#CC1(NC(=O)[C@H](Cc2cc(Cl)c(O)c(Cl)c2)N[C@@H](c2ccc(N3CCN(CC(F)(F)F)CC3)cc2)C(F)(F)F)CC1 ; N#CC1(NC(=O)[C@H](Cc2cc(Cl)c(O)c(Cl)c2)N[C@@H](c2ccc(-c3nnnn3C3CC3)cc2)C(F)(F)C(F)(F)F)CC1 ; CCC[C@H](NC(=O)[C@H](Cc1nc[nH]n1)N[C@@H](c1ccc(-c2nnnn2C2CC2)cc1)C(F)F)C(=O)C(=O)NC1CC1 ; N#CC1(NC(=O)[C@H](Cc2nc[nH]n2)N[C@@H](c2ccc(OC3CCCC3)c(F)c2)C(F)(F)F)CC1 ; N#CC1(NC(=O)[C@H](Cc2nc[nH]n2)N[C@@H](c2ccc(N3CCN(CC(F)(F)F)CC3)cc2)C(F)(F)F)CC1]

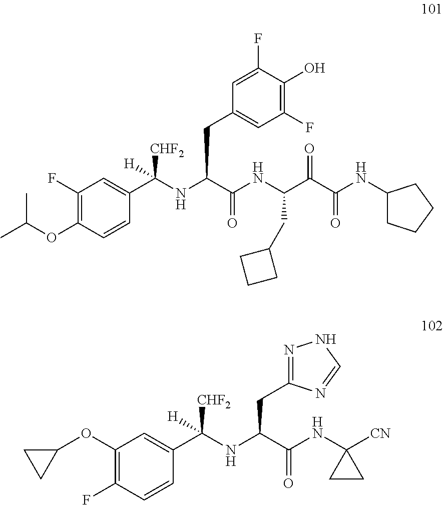 [SMILES: CC(C)Oc1ccc([C@H](N[C@@H](Cc2cc(F)c(O)c(F)c2)C(=O)N[C@@H](CC2CCC2)C(=O)C(=O)NC2CCCC2)C(F)F)cc1F ; N#CC1(NC(=O)[C@H](Cc2nc[nH]n2)N[C@@H](c2ccc(F)c(OC3CC3)c2)C(F)F)CC1]

The above compounds 1—are disclosed herein as individual compounds or any combinations of individual compounds thereof.

### Biological Examples

The following illustrates the testing of compounds of the invention.

### Example 6

### Cathepsin B Assay

Solutions of test compounds in varying concentrations were prepared in 10 μL of dimethyl sulfoxide (DMSO) and then diluted into assay buffer (40 μL, comprising: N,N-bis(2-hydroxyethyl)-2-aminoethanesulfonic acid (BES), 50 mM (pH 6); polyoxyethylenesorbitan monolaurate, 0.05%; and dithiothreitol (DTT), 2.5 mM). Human cathepsin B (0.025 pMoles in 25 μL of assay buffer) was added to the dilutions. The assay solutions were mixed for 5-10 seconds on a shaker plate, covered and incubated for 30 minutes at ambient temperature. Boc-Leu-Arg-Arg-AMC (20 μM in 1% DMSO) was added to the assay solutions and hydrolysis was followed spectrophotometrically at (A 460 nm) for 5 minutes. Apparent inhibition constants (K) were calculated from the enzyme progress curves using standard mathematical models.

Compounds of the invention were tested by the above-described assay and observed to exhibit cathepsin B inhibitory activity.

| Compound | cat B Kᵢ | |
|---|---|---|
| N-cyanomethyl-2S-[2,2,2-trifluoro-1S-(4-bromophenyl)- | 3.5 | nM |
| ethylamino]-3-(3,5-diiodo-4-hydroxyphenyl)-propanamide | | |
| N-(1-cyanocyclopropyl)-2S-[2,2,2-trifluoro-1S-(4- | 13 | nM |
| bromophenyl)-ethylamino]-3-(3,5-diiodo-4-hydroxyphenyl)- | | |
| propanamide | | |
| N-(1-cyanocyclopropyl)-2S-[2,2,2-trifluoro-1S-(4- | 45 | nM |
| bromophenyl)-ethylamino]-3-(3,5-dichloro-4-hydroxyphenyl)- | | |
| propanamide | | |
| N-(1-cyanocyclopropyl)-2S-[2,2,2-trifluoro-1S-(4- | 130 | nM |
| fluorophenyl)-ethylamino]-3-(3,5-dichloro-4-hydroxyphenyl)- | | |
| propanamide | | |
| N-cyclopropyl-3S-{3-[3,5-diiodo-4-hydroxyphenyl)-2S-(2,2,2- | 1.28 | nM |
| trifluoro)-1S-(4-bromophenyl)ethylamino]propionylamino}- | | |
| 2-oxopentanamide | | |
| N-cyclopropyl-3S-{3-[3,5-diiodo-4-hydroxyphenyl)-2S-(2,2,2- | 1.16 | nM |
| trifluoro)-1S-(4-fluorophenyl)ethylamino]propionylamino}- | | |
| 2-oxopentanamide | | |
| N-cyclopropyl-3S-{3-[3,5-dichloro-4-hydroxyphenyl)-2S- | ≦1 | nM |
| (2,2,2-trifluoro)-1S-(4-fluorophenyl)ethylamino]propionyl- | | |
| amino}-2-oxopentanamide | | |
| N-cyclopropyl-3S-{3-[3,5-dichloro-4-hydroxyphenyl)-2S- | 1.53 | nM |
| (2,2,2-trifluoro)-1S-(4-bromophenyl)ethylamino]propionyl- | | |
| amino}-2-oxopentanamide | | |

### Example 7

### Cathepsin K Assay

Solutions of test compounds in varying concentrations were prepared in 10 μL of dimethyl sulfoxide (DMSO) and then diluted into assay buffer (40 mL, comprising: MES, 50 mM (pH 5.5); EDTA, 2.5 mM; and DTT, 2.5 mM). Human cathepsin K (0.0906 pMoles in 25 μL of assay buffer) was added to the dilutions. The assay solutions were mixed for 5-10 seconds on a shaker plate, covered and incubated for 30 min at ambient temperature. Z-Phe-Arg-AMC (4 nMoles in 25 μL of assay buffer) was added to the assay solutions and hydrolysis was followed spectrophotometrically at (A 460 nm) for 5 min. Apparent inhibition constants (Kᵢ) were calculated from the enzyme progress curves using standard mathematical models.

Compounds of the invention were tested by the above-described assay and observed to exhibit cathepsin K inhibitory activity.

### Example 8

### Cathepsin L Assay

Solutions of test compounds in varying concentrations were prepared in 10 μL of dimethyl sulfoxide (DMSO) and then diluted into assay buffer (40 mL, comprising: MES, 50 mM (pH 5.5); EDTA, 2.5 mM; and DTT, 2.5 mM). Human cathepsin L (0.05 pMoles in 25 of assay buffer) was added to the dilutions. The assay solutions were mixed for 5-10 seconds on a shaker plate, covered and incubated for 30 minutes at ambient temperature. Z-Phe-Arg-AMC (1 nMoles in 25 μL of assay buffer) was added to the assay solutions and hydrolysis was followed spectrophotometrically at (λ 460 nm) for 5 minutes. Apparent inhibition constants (Kᵢ) were calculated from the enzyme progress curves using standard mathematical models.

Compounds of the invention were tested by the above-described or similar assay and observed to exhibit cathepsin L inhibitory activity.

### Example 9

### Cathepsin S Assay

Solutions of test compounds in varying concentrations were prepared in 10 μL of dimethyl sulfoxide (DMSO) and then diluted into assay buffer (40 mL, comprising: MES, 50 mM (pH 6.5); EDTA, 2.5 mM; and NaCl, 100 mM); P-mercaptoethanol, 2.5 mM; and BSA, 0.00%. Human cathepsin S (0.05 pMoles in 25 μL of assay buffer) was added to the dilutions. The assay solutions were mixed for 5-10 seconds on a shaker plate, covered and incubated for 30 minutes at ambient temperature. Z-Val-Val-Arg-AMC (4 nMoles in 25 μL of assay buffer containing 10% DMSO) was added to the assay solutions and hydrolysis was followed spectrophotometrically (at λ 460 nm) for 5 min. Apparent inhibition constants (Kᵢ) were calculated from the enzyme progress curves using standard mathematical models.

Compounds of the invention were tested by the above-described or similar assay and observed to exhibit cathepsin S inhibitory activity.

### Example 10

### Cathepsin F Assay

Solutions of test compounds in varying concentrations were prepared in 10 μL of dimethyl sulfoxide (DMSO) and then diluted into assay buffer (40 μL, comprising: MES, 50 mM (pH 6.5); EDTA, 2.5 mM; and NaCl, 100 mM); DTT, 2.5 mM; and BSA, 0.01%. Human cathepsin F (0.1 pMoles in 25 μL of assay buffer) was added to the dilutions. The assay solutions were mixed for 5-10 seconds on a shaker plate, covered and incubated for 30 minutes at ambient temperature. Z-Phe-Arg-AMC (2 nMoles in 25 μL of assay buffer containing 10% DMSO) was added to the assay solutions and hydrolysis was followed spectrophotometrically (at λ 460 μm) for 5 minutes. Apparent inhibition constants (Kᵢ) were calculated from the enzyme progress curves using standard mathematical models.

Compounds of the invention were tested by the above-described or similar assay and observed to exhibit cathepsin F inhibitory activity.

### Formulation Examples

The following illustrates the formulations of the compounds of the invention.

### Example 11

### Representative Pharmaceutical Formulations Containing a Compound of the Invention

TABLE 1

| | | |
|---|---|---|
| compound of the invention | 10-100 | mg |
| citric acid monohydrate | 105 | mg |
| sodium hydroxide | 18 | mg |

| | |
|---|---|
| flavoring | |
| water | q.s. to 100 mL |

TABLE 2
Intravenous Formulation

| | |
|---|---|
| compound of the invention | 0.1-10 mg |
| dextrose monohydrate | q.s. to make isotonic |
| citric acid monohydrate | 1.05 mg |
| sodium hydroxide | 0.18 mg |
| saline | q.s. to 1.0 mL |

TABLE 3
Tablet Formulation

| | |
|---|---|
| compound of the invention | 1% |
| microcrystalline cellulose | 73% |
| steric acid | 25% |
| colloidal silica | 1% |

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

1. A compound of Formula I: 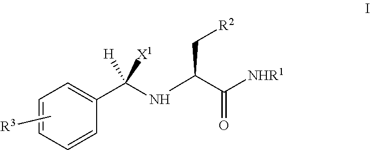
in which:
X¹ is —CF₃, —CF₂CF₃ or —CHF₂;
R¹ is a group of Formula (a), (b) or (c): 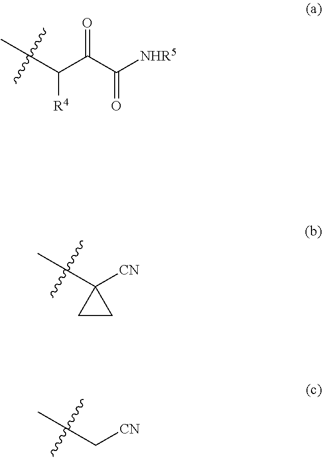
where R⁴ is (C₁₋₃)-n-alkyl or (C₃₋₄)cycloalkylmethyl and R⁵ is hydrogen, (C₁₋₃)alkyl or (C₃₋₇)cycloalkyl;
R² is a group of Formula (d), (e) or (f): 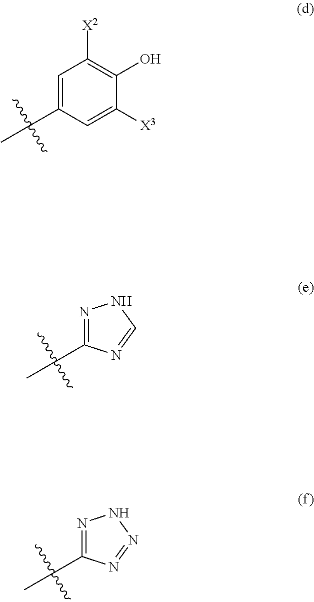
where X² and X³ are independently fluoro, iodo, bromo or chloro; and
R³ is one to three substituents selected from hydrogen, C₁₋₆alkoxy, C₃₋₆cycloalkoxy, fluoro, chloro, bromo, trifluoromethyl, trifluoromethoxy, heteroaryl or heterocyclyl, wherein the heteroaryl and heterocyclyl may be further substituted with 2,2,2-trifluoroethyl, (C₁₋₆)alkyl, or (C₃₋₆)cycloalkyl; and the pharmaceutically acceptable salts thereof.

2. A pharmaceutical composition comprising a compound of claim 1 or a pharmaceutically acceptable salt thereof and a pharmaceutically acceptable excipient.

3. A method for treating a disease in an animal mediated by cysteine proteases which method comprises administering to the animal a pharmaceutical composition comprising a therapeutically effective amount of a compound of claim 1 or a pharmaceutically acceptable salt thereof and a pharmaceutically acceptable excipient.

4. The method of claim 3 in which the disease is selected from the group consisting of arthritis, muscular dystrophy, inflammation, tumor invasion, glomerulonephritis, periodontal disease, tumor invasion and metastasis, Alzheimer's Disease inflammatory diseases such as chronic and acute pancreatitis, inflammatory airway disease, bone and joint disorders, osteoporosis, osteoarthritis, rheumatoid arthritis, psoriasis, autoimmune disorders, fibrotic disease, HCV-associated liver fibrosis, steatosis, alcohol-associated steatohepatitis, non-alcoholic fatty liver disease, pulmonary fibrosis, idiopathic pulmonary fibrosis, pathological diagnosis of interstitial pneumonia following lung biopsy, renal fibrosis, cardiac fibrosis, retinal angiogenesis, fibrosis/gliosis in the eye, scleroderma, systemic sclerosis and keloids and other forms of scarring.

5. The method of claim 3 in which the compound is selected from:
N-cyanomethyl-2S-[2,2,2-trifluoro-1S-(4-bromophenyl)-ethylamino]-3-(3,5-diiodo-4-hydroxyphenyl)-propanamide;
N-(1-cyanocyclopropyl)-2S-[2,2,2-trifluoro-1S-(4-bromophenyl)-ethylamino]-3-(3,5-diiodo-4-hydroxyphenyl)-propanamide;
N-(1-cyanocyclopropyl)-2S-[2,2,2-trifluoro-1S-(4-bromophenyl)-ethylamino]-3-(3,5-dichloro-4-hydroxyphenyl)-propanamide;
N-(1-cyanocyclopropyl)-2S-[2,2,2-trifluoro-1S-(4-fluorophenyl)-ethylamino]-3-(3,5-dichloro-4-hydroxyphenyl)-propanamide;
N-cyclopropyl-3S-{3-[3,5-diiodo-4-hydroxyphenyl-2S-(2,2,2-trifluoro)-1S-(4-bromophenyl)ethylamino]propionylamino}-2-oxopentanamide;
N-cyclopropyl-3S-[3-[3,5-diiodo-4-hydroxyphenyl-2S-(2,2,2-trifluoro)-1S-(4-fluorophenyl)ethylamino]propionylamino]-2-oxopentanamide;
N-cyclopropyl-3S-{3-[3,5-dichloro-4-hydroxyphenyl-2S-(2,2,2-trifluoro)-1S-(4-fluorophenyl)ethylamino]propionylamino}-2-oxopentanamide; and
N-cyclopropyl-3S-{3-[3,5-dichloro-4-hydroxyphenyl-2S-(2,2,2-trifluoro)-1S-(4-bromophenyl)ethylamino]propionylamino}-2-oxopentanamide.

6. A method of treating liver fibrosis associated with non-alcoholic fatty liver disease in a human patient, which process comprises administering to the patient a pharmaceutical composition comprising a therapeutically effective amount of a compound of claim 1 or a pharmaceutically acceptable salt thereof and a pharmaceutically acceptable excipient.

7. The method of claim 6 in which the compound is selected from:
N-cyanomethyl-2S-[2,2,2-trifluoro-1S-(4-bromophenyl)-ethylamino]-3-(3,5-diiodo-4-hydroxyphenyl)-propanamide;
N-(1-cyanocyclopropyl)-2S-[2,2,2-trifluoro-1S-(4-bromophenyl)-ethylamino]-3-(3,5-diiodo-4-hydroxyphenyl)-propanamide;
N-(1-cyanocyclopropyl)-2S-[2,2,2-trifluoro-1S-(4-bromophenyl)-ethylamino]-3-(3,5-dichloro-4-hydroxyphenyl)-propanamide;
N-(1-cyanocyclopropyl)-2S-[2,2,2-trifluoro-1S-(4-fluorophenyl)-ethylamino]-3-(3,5-dichloro-4-hydroxyphenyl)-propanamide;
N-cyclopropyl-3S-{3-[3,5-diiodo-4-hydroxyphenyl-2S-(2,2,2-trifluoro)-1S-(4-bromophenyl)ethylamino]propionylamino}-2-oxopentanamide;
N-cyclopropyl-3S-{3-[3,5-diiodo-4-hydroxyphenyl-2S-(2,2,2-trifluoro)-1S-(4-fluorophenyl)ethylamino]propionylamino}-2-oxopentanamide;
N-cyclopropyl-3S-{3-[3,5-dichloro-4-hydroxyphenyl-2S-(2,2,2-trifluoro)-1S-(4-fluorophenyl)ethylamino]propionylamino}-2-oxopentanamide; or
N-cyclopropyl-3S-{3-[3,5-dichloro-4-hydroxyphenyl-2S-(2,2,2-trifluoro)-1S-(4-bromophenyl)ethylamino]propionylamino}-2-oxopentanamide.

8. A method of treating acute pancreatitis in a human patient, which process comprises administering to the patient a pharmaceutical composition comprising a therapeutically effective amount of a compound claim 1 or a pharmaceutically acceptable salt thereof and a pharmaceutically acceptable excipient.

9. The method of claim 8 in which the compound is selected from:
N-cyanomethyl-2S-[2,2,2-trifluoro-1S-(4-bromophenyl)-ethylamino]-3-(3,5-diiodo-4-hydroxyphenyl)-propanamide;
N-(1-cyanocyclopropyl)-2S-[2,2,2-trifluoro-1S-(4-bromophenyl)-ethylamino]-3-(3,5-diiodo-4-hydroxyphenyl)-propanamide;
N-(1-cyanocyclopropyl)-2S-[2,2,2-trifluoro-1S-(4-bromophenyl)-ethylamino]-3-(3,5-dichloro-4-hydroxyphenyl)-propanamide;
N-(1-cyanocyclopropyl)-2S-[2,2,2-trifluoro-1S-(4-fluorophenyl)-ethylamino]-3-(3,5-dichloro-4-hydroxyphenyl)-propanamide;
N-cyclopropyl-3S-{3-[3,5-diiodo-4-hydroxyphenyl)-2S-(2,2,2-trifluoro)-1S-(4-bromophenyl)ethylamino]propionylamino}-2-oxopentanamide;
N-cyclopropyl-3S-{3-[3,5-diiodo-4-hydroxyphenyl)-2S-(2,2,2-trifluoro)-1S-(4-fluorophenyl)ethylamino]propionylamino}-2-oxopentanamide;
N-cyclopropyl-3S-{3-[3,5-dichloro-4-hydroxyphenyl)-2S-(2,2,2-trifluoro)-1S-(4-fluorophenyl)ethylamino]propionylamino}-2-oxopentanamide; or
N-cyclopropyl-3S-{3-[3,5-dichloro-4-hydroxyphenyl)-2S-(2,2,2-trifluoro)-1S-(4-bromophenyl)ethylamino]propionylamino}-2-oxopentanamide.

10. The method of claim 4 in which the compound is selected from:
N-cyanomethyl-2S-[2,2,2-trifluoro-1S-(4-bromophenyl)-ethylamino]-3-(3,5-diiodo-4-hydroxyphenyl)-propanamide;
N-(1-cyanocyclopropyl)-2S-[2,2,2-trifluoro-1S-(4-bromophenyl)-ethylamino]-3-(3,5-diiodo-4-hydroxyphenyl)-propanamide;
N-(1-cyanocyclopropyl)-2S-[2,2,2-trifluoro-1S-(4-bromophenyl)-ethylamino]-3-(3,5-dichloro-4-hydroxyphenyl)-propanamide;
N-(1-cyanocyclopropyl)-2S-[2,2,2-trifluoro-1S-(4-fluorophenyl)-ethylamino]-3-(3,5-dichloro-4-hydroxyphenyl)-propanamide;
N-cyclopropyl-3S-{3-[3,5-diiodo-4-hydroxyphenyl)-2S-(2,2,2-trifluoro)-1S-(4-bromophenyl)ethylamino]propionylamino}-2-oxopentanamide;
N-cyclopropyl-3S-{3-[3,5-diiodo-4-hydroxyphenyl)-2S-(2,2,2-trifluoro)-1S-(4-fluorophenyl)ethylamino]propionylamino}-2-oxopentanamide;
N-cyclopropyl-3S-{3-[3,5-dichloro-4-hydroxyphenyl)-2S-(2,2,2-trifluoro)-1S-(4-fluorophenyl)ethylamino]propionylamino}-2-oxopentanamide; or
N-cyclopropyl-3S-{3-[3,5-dichloro-4-hydroxyphenyl)-2S-(2,2,2-trifluoro)-1S-(4-bromophenyl)ethylamino]propionylamino}-2-oxopentanamide.

11. A process for preparing a compound of Formula I: 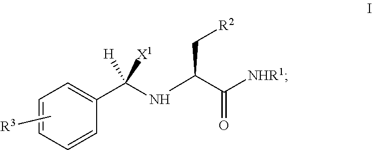
wherein R¹ is a group of Formula (a), (b) or (c), which process comprises contacting a compound of Formula 4: 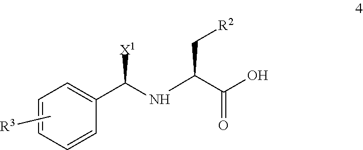
with a compound having the formula NH₂R⁶, where R⁶ is group of Formula (a), (b) or (c), in the presence of a suitable coupling agent and base, wherein the compound of Formula 4 is prepared by reducing the compound of Formula 3: 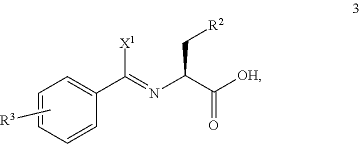
wherein the compound of Formula 3 is prepared by contacting a compound of Formula 1: 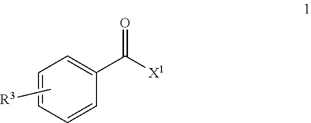
with a compound of Formula 2: 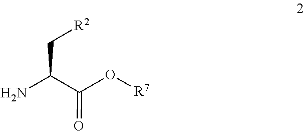
in which R⁷ is C₁₋₇alkyl or C₃₋₆cycloalkylmethyl, in the presence of a weak base.

12. The compound of claim 1 in which R¹ is a group of Formula (b) or (c).

13. The compound of claim 1 in which R² is a group of Formula (d).

14. The compound of claim 1 selected from:
N-cyanomethyl-2S-[2,2,2-trifluoro-1S-(4-bromophenyl)-ethylamino]-3-(3,5-diiodo-4-hydroxyphenyl)-propanamide;
N-(1-cyanocyclopropyl)-2S-[2,2,2-trifluoro-1S-(4-bromophenyl)-ethylamino]-3-(3,5-diiodo-4-hydroxyphenyl)-propanamide;
N-(1-cyanocyclopropyl)-2S-[2,2,2-trifluoro-1S-(4-bromophenyl)-ethylamino]-3-(3,5-dichloro-4-hydroxyphenyl)-propanamide; or
N-(1-cyanocyclopropyl)-2S-[2,2,2-trifluoro-1S-(4-fluorophenyl)-ethylamino]-3-(3,5-dichloro-4-hydroxyphenyl)-propanamide.

15. The compound of claim 1 in which R¹ is a group of Formula (a).

16. The process of claim 11 in which R² is a group of Formula (d).

17. The compound of claim 1 selected from:
N-cyclopropyl-3S-{3-[3,5-diiodo-4-hydroxyphenyl)-2S-(2,2,2-trifluoro)-1S-(4-bromophenyl)ethylamino]propionylamino}-2-oxopentanamide;
N-cyclopropyl-3S-{3-[3,5-diiodo-4-hydroxyphenyl)-2S-(2,2,2-trifluoro)-1S-(4-fluorophenyl)ethylamino]propionylamino}-2-oxopentanamide;
N-cyclopropyl-3S-{3-[3,5-dichloro-4-hydroxyphenyl)-2S-(2,2,2-trifluoro)-1S-(4-fluorophenyl)ethylamino]propionylamino}-2-oxopentanamide; or
N-cyclopropyl-3S-{3-[3,5-dichloro-4-hydroxyphenyl)-2S-(2,2,2-trifluoro)-1S-(4-bromophenyl)ethylamino]propionylamino}-2-oxopentanamide.

18. The compound of claim 1 selected from the following formulae: 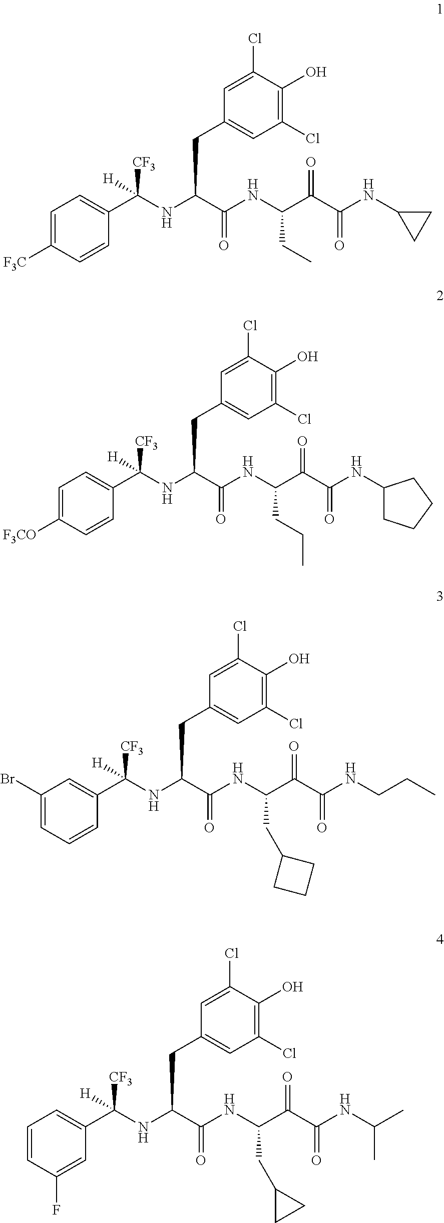 [SMILES: CC[C@H](NC(=O)[C@H](Cc1cc(Cl)c(O)c(Cl)c1)N[C@@H](c1ccc(C(F)(F)F)cc1)C(F)(F)F)C(=O)C(=O)NC1CC1 ; CCC[C@H](NC(=O)[C@H](Cc1cc(Cl)c(O)c(Cl)c1)N[C@@H](c1ccc(OC(F)(F)F)cc1)C(F)(F)F)C(=O)C(=O)NC1CCCC1 ; CCCNC(=O)C(=O)[C@H](CC1CCC1)NC(=O)[C@H](Cc1cc(Cl)c(O)c(Cl)c1)N[C@@H](c1cccc(Br)c1)C(F)(F)F ; CC(C)NC(=O)C(=O)[C@H](CC1CC1)NC(=O)[C@H](Cc1cc(Cl)c(O)c(Cl)c1)N[C@@H](c1cccc(F)c1)C(F)(F)F] 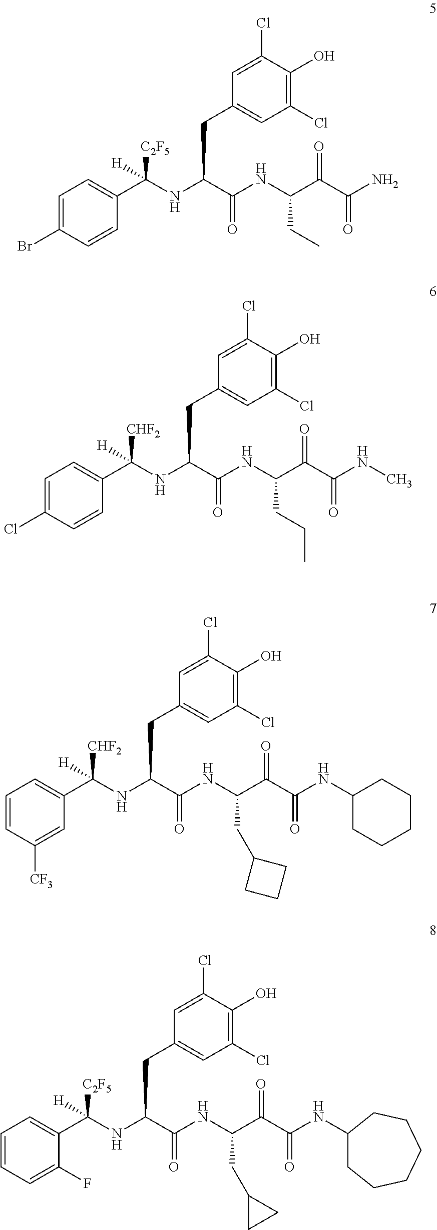 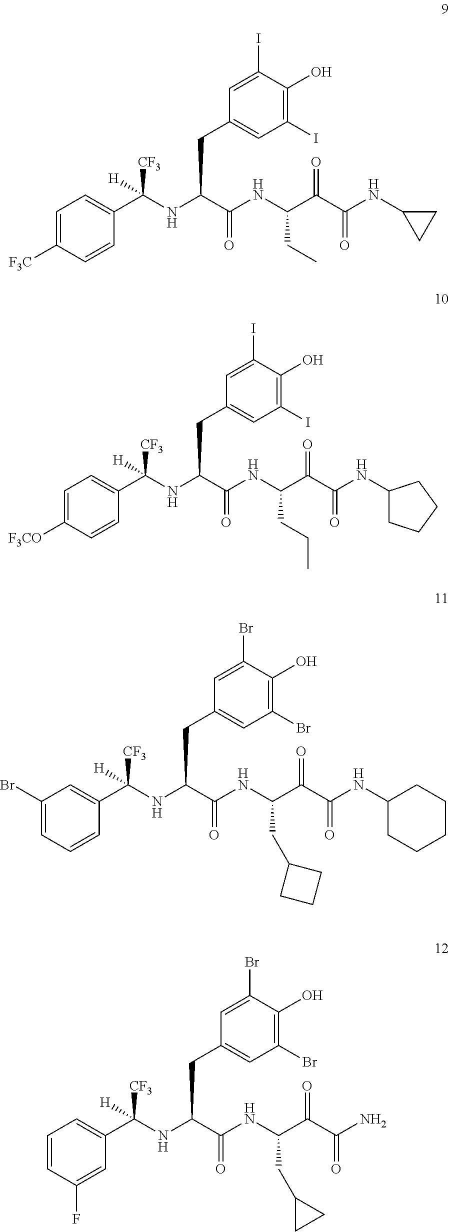 [SMILES: CC[C@H](NC(=O)[C@H](Cc1cc(I)c(O)c(I)c1)N[C@@H](c1ccc(C(F)(F)F)cc1)C(F)(F)F)C(=O)C(=O)NC1CC1 ; CCC[C@H](NC(=O)[C@H](Cc1cc(I)c(O)c(I)c1)N[C@@H](c1ccc(OC(F)(F)F)cc1)C(F)(F)F)C(=O)C(=O)NC1CCCC1 ; O=C(NC1CCCCC1)C(=O)[C@H](CC1CCC1)NC(=O)[C@H](Cc1cc(Br)c(O)c(Br)c1)N[C@@H](c1cccc(Br)c1)C(F)(F)F ; NC(=O)C(=O)[C@H](CC1CC1)NC(=O)[C@H](Cc1cc(Br)c(O)c(Br)c1)N[C@@H](c1cccc(F)c1)C(F)(F)F] 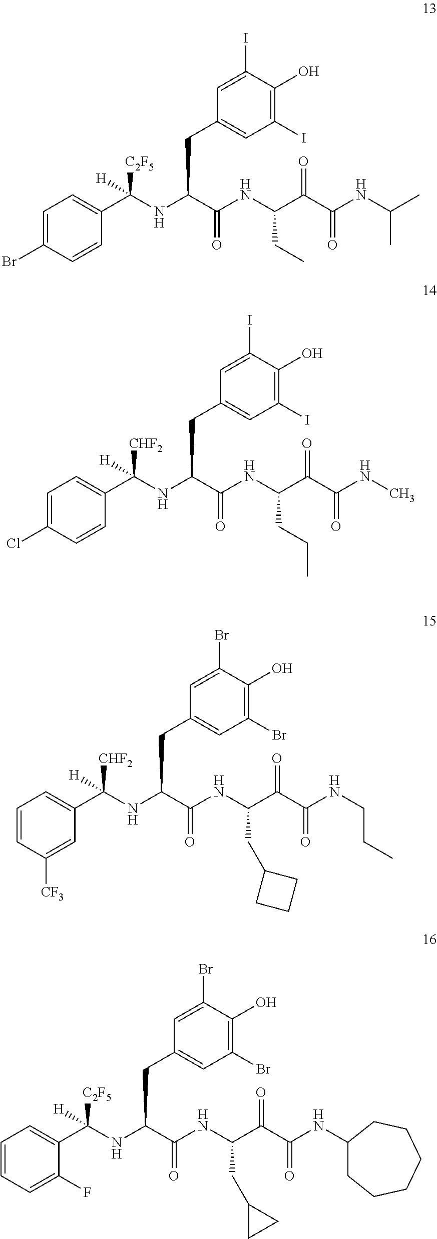 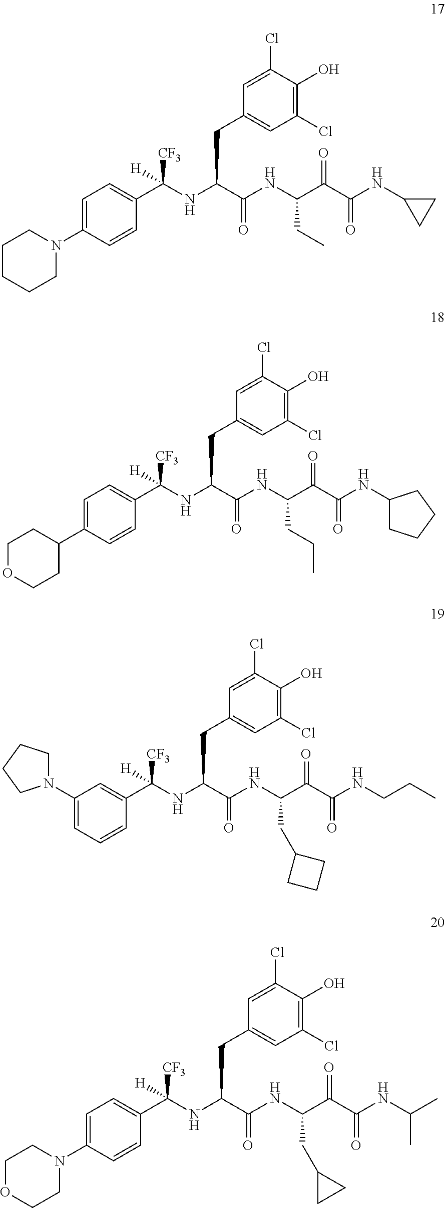 [SMILES: CC[C@H](NC(=O)[C@H](Cc1cc(Cl)c(O)c(Cl)c1)N[C@@H](c1ccc(N2CCCCC2)cc1)C(F)(F)F)C(=O)C(=O)NC1CC1 ; CCC[C@H](NC(=O)[C@H](Cc1cc(Cl)c(O)c(Cl)c1)N[C@@H](c1ccc(C2CCOCC2)cc1)C(F)(F)F)C(=O)C(=O)NC1CCCC1 ; CCCNC(=O)C(=O)[C@H](CC1CCC1)NC(=O)[C@H](Cc1cc(Cl)c(O)c(Cl)c1)N[C@@H](c1cccc(N2CCCC2)c1)C(F)(F)F ; CC(C)NC(=O)C(=O)[C@H](CC1CC1)NC(=O)[C@H](Cc1cc(Cl)c(O)c(Cl)c1)N[C@@H](c1ccc(N2CCOCC2)cc1)C(F)(F)F] 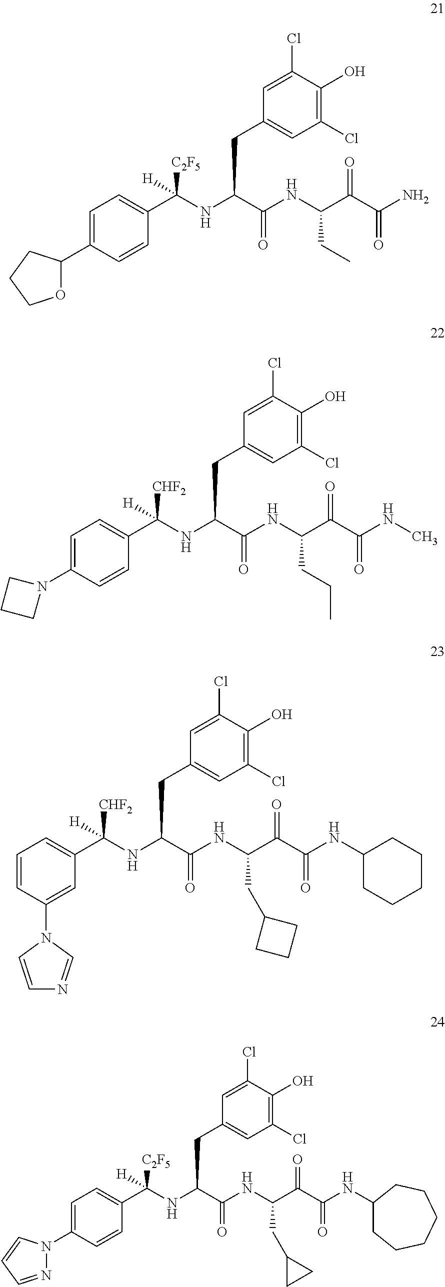 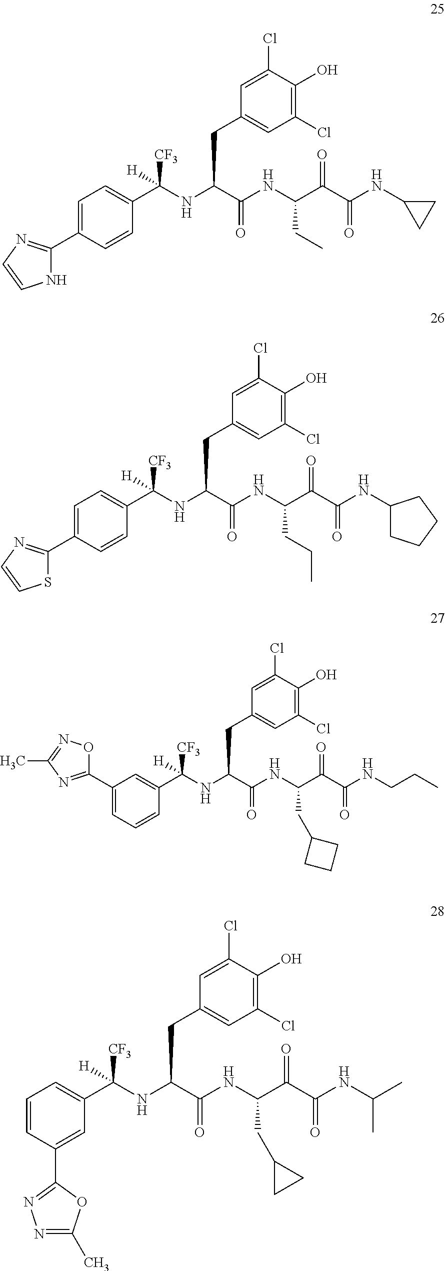 [SMILES: CC[C@H](NC(=O)[C@H](Cc1cc(Cl)c(O)c(Cl)c1)N[C@@H](c1ccc(-c2ncc[nH]2)cc1)C(F)(F)F)C(=O)C(=O)NC1CC1 ; CCC[C@H](NC(=O)[C@H](Cc1cc(Cl)c(O)c(Cl)c1)N[C@@H](c1ccc(-c2nccs2)cc1)C(F)(F)F)C(=O)C(=O)NC1CCCC1 ; CCCNC(=O)C(=O)[C@H](CC1CCC1)NC(=O)[C@H](Cc1cc(Cl)c(O)c(Cl)c1)N[C@@H](c1cccc(-c2nc(C)no2)c1)C(F)(F)F ; Cc1nnc(-c2cccc([C@H](N[C@@H](Cc3cc(Cl)c(O)c(Cl)c3)C(=O)N[C@@H](CC3CC3)C(=O)C(=O)NC(C)C)C(F)(F)F)c2)o1] 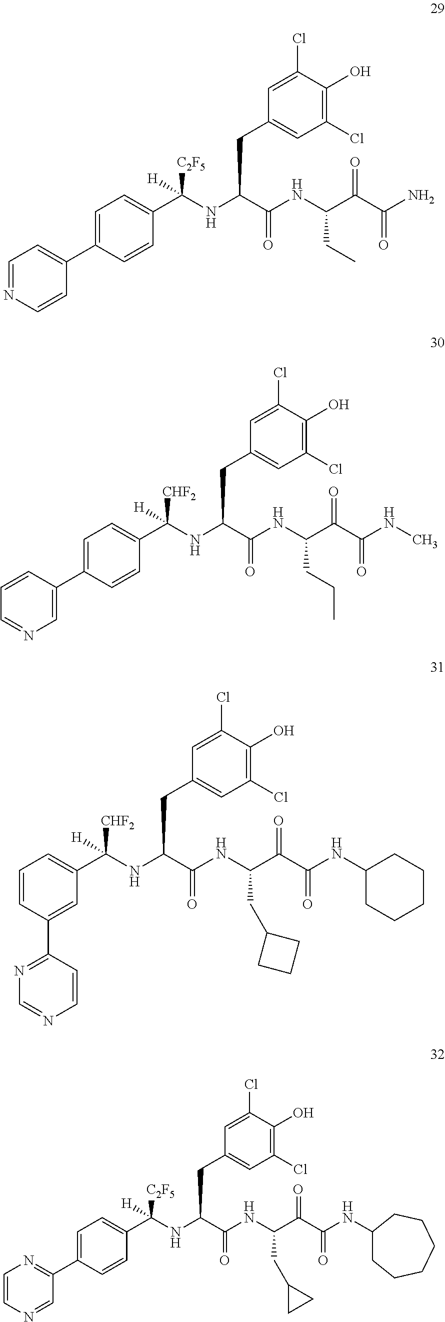 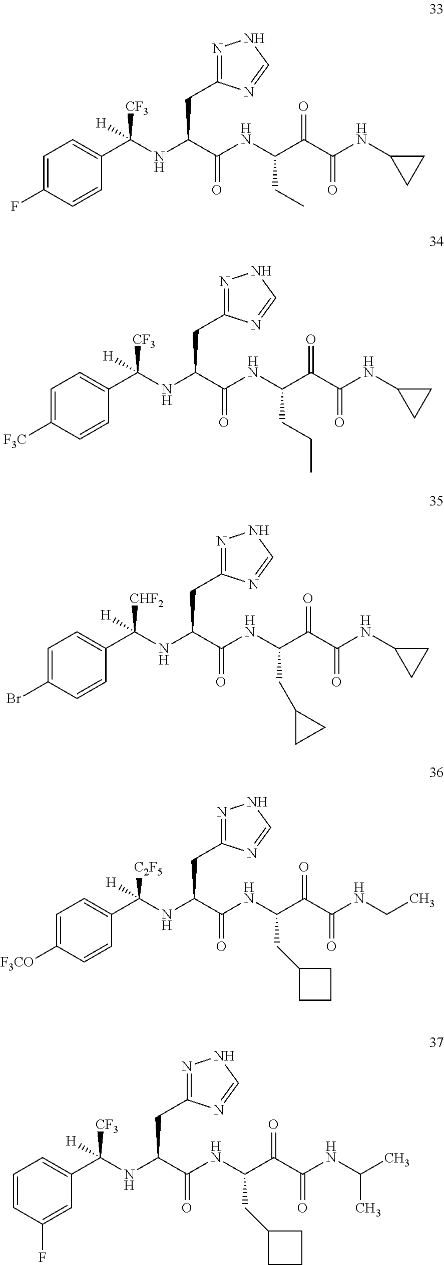 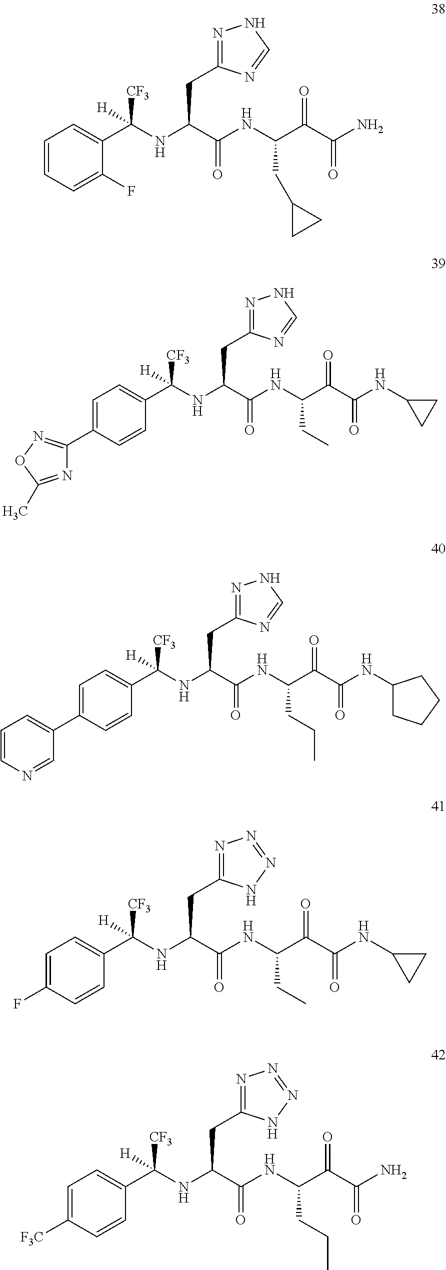 [SMILES: NC(=O)C(=O)[C@H](CC1CC1)NC(=O)[C@H](Cc1nc[nH]n1)N[C@@H](c1ccccc1F)C(F)(F)F ; CC[C@H](NC(=O)[C@H](Cc1nc[nH]n1)N[C@@H](c1ccc(-c2noc(C)n2)cc1)C(F)(F)F)C(=O)C(=O)NC1CC1 ; CCC[C@H](NC(=O)[C@H](Cc1nc[nH]n1)N[C@@H](c1ccc(-c2cccnc2)cc1)C(F)(F)F)C(=O)C(=O)NC1CCCC1 ; CC[C@H](NC(=O)[C@H](Cc1nnn[nH]1)N[C@@H](c1ccc(F)cc1)C(F)(F)F)C(=O)C(=O)NC1CC1 ; CCC[C@H](NC(=O)[C@H](Cc1nnn[nH]1)N[C@@H](c1ccc(C(F)(F)F)cc1)C(F)(F)F)C(=O)C(N)=O] 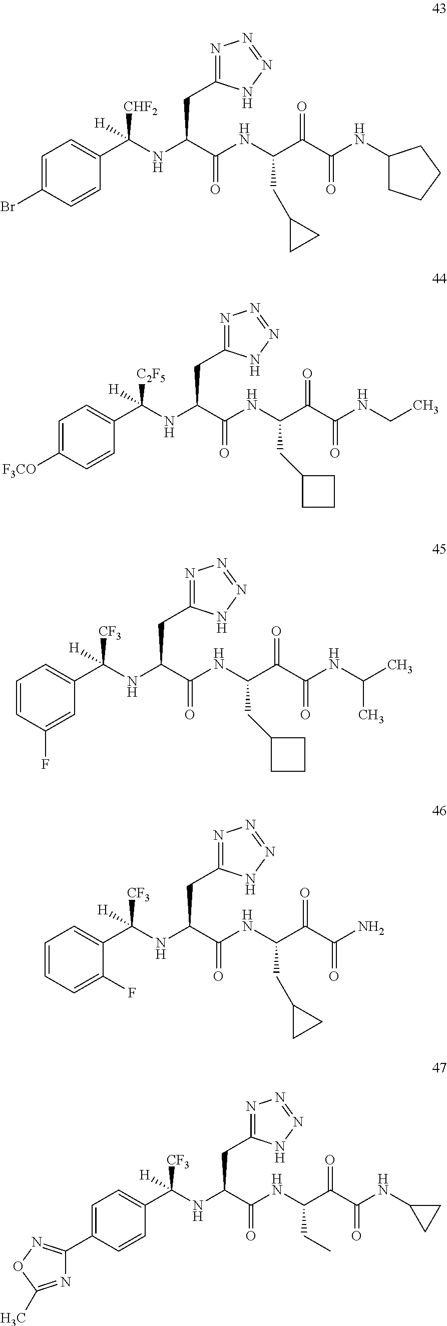 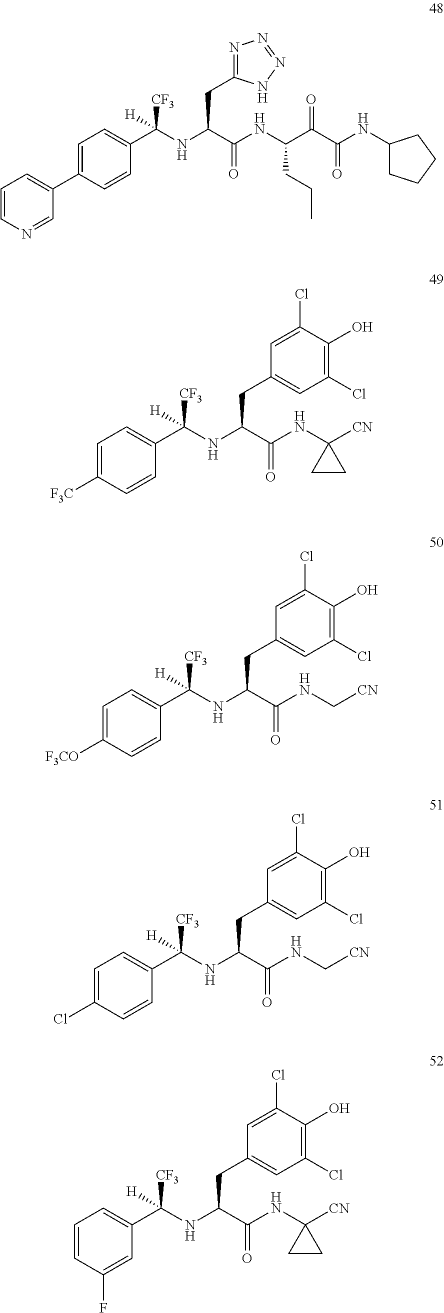 [SMILES: CCC[C@H](NC(=O)[C@H](Cc1nnn[nH]1)N[C@@H](c1ccc(-c2cccnc2)cc1)C(F)(F)F)C(=O)C(=O)NC1CCCC1 ; N#CC1(NC(=O)[C@H](Cc2cc(Cl)c(O)c(Cl)c2)N[C@@H](c2ccc(C(F)(F)F)cc2)C(F)(F)F)CC1 ; N#CCNC(=O)[C@H](Cc1cc(Cl)c(O)c(Cl)c1)N[C@@H](c1ccc(OC(F)(F)F)cc1)C(F)(F)F ; N#CCNC(=O)[C@H](Cc1cc(Cl)c(O)c(Cl)c1)N[C@@H](c1ccc(Cl)cc1)C(F)(F)F ; N#CC1(NC(=O)[C@H](Cc2cc(Cl)c(O)c(Cl)c2)N[C@@H](c2cccc(F)c2)C(F)(F)F)CC1] 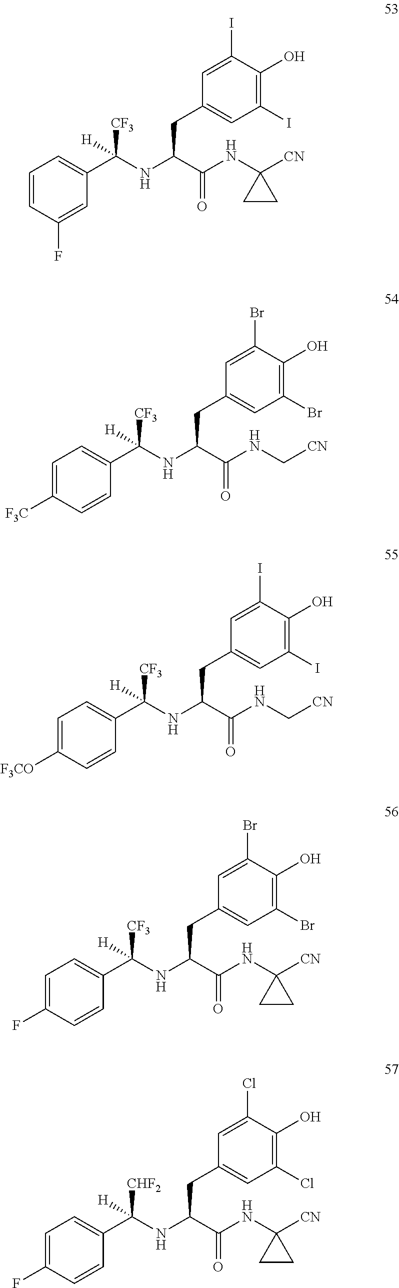 [SMILES: N#CC1(NC(=O)[C@H](Cc2cc(I)c(O)c(I)c2)N[C@@H](c2cccc(F)c2)C(F)(F)F)CC1 ; N#CCNC(=O)[C@H](Cc1cc(Br)c(O)c(Br)c1)N[C@@H](c1ccc(C(F)(F)F)cc1)C(F)(F)F ; N#CCNC(=O)[C@H](Cc1cc(I)c(O)c(I)c1)N[C@@H](c1ccc(OC(F)(F)F)cc1)C(F)(F)F ; N#CC1(NC(=O)[C@H](Cc2cc(Br)c(O)c(Br)c2)N[C@@H](c2ccc(F)cc2)C(F)(F)F)CC1 ; N#CC1(NC(=O)[C@H](Cc2cc(Cl)c(O)c(Cl)c2)N[C@@H](c2ccc(F)cc2)C(F)F)CC1] 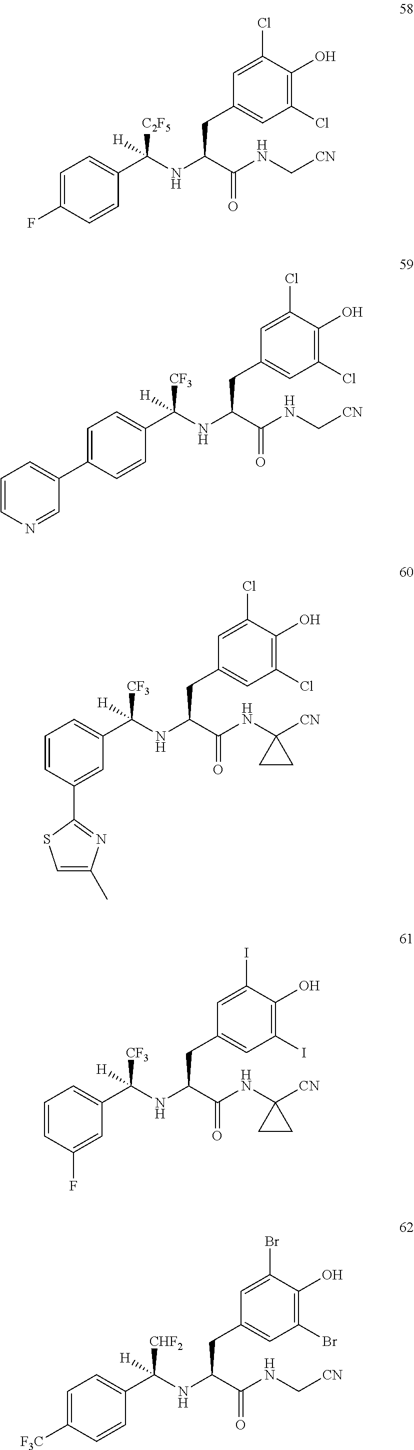 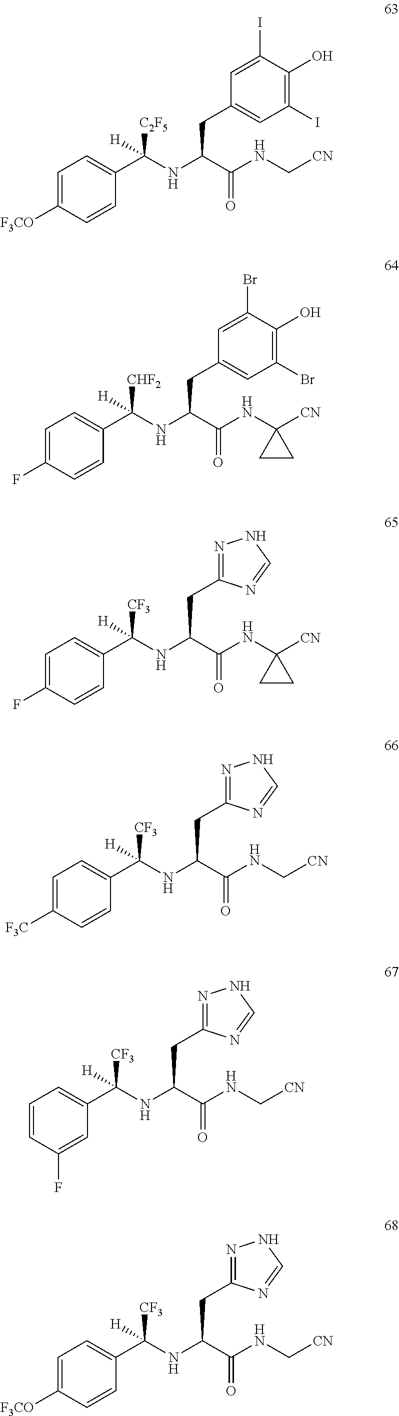 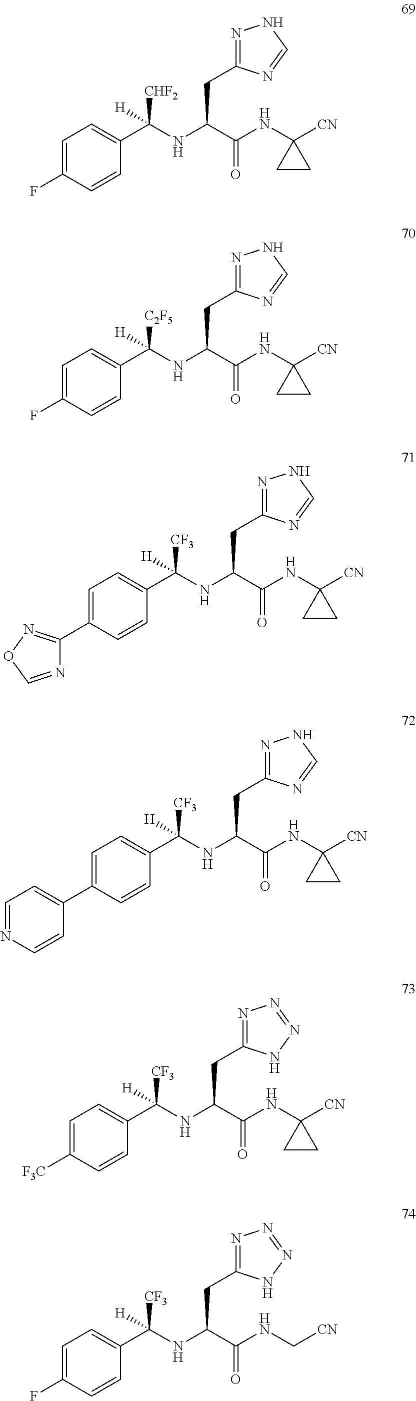 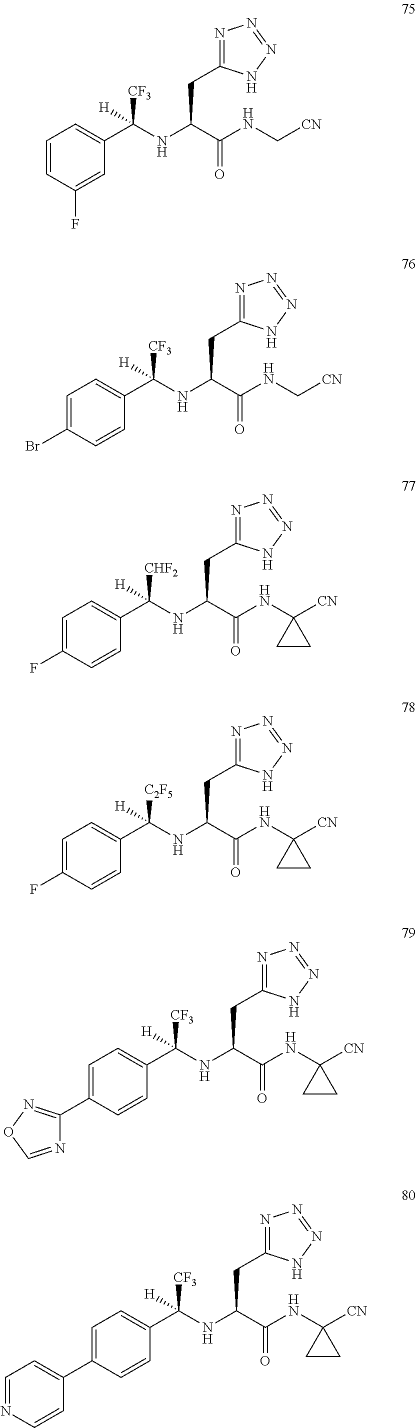 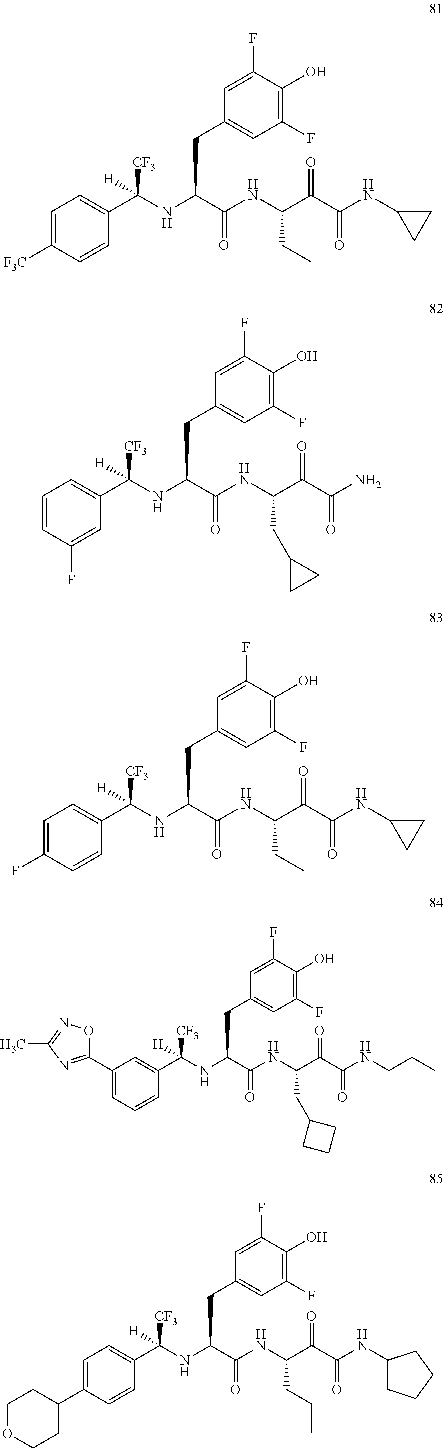 [SMILES: CC[C@H](NC(=O)[C@H](Cc1cc(F)c(O)c(F)c1)N[C@@H](c1ccc(C(F)(F)F)cc1)C(F)(F)F)C(=O)C(=O)NC1CC1 ; NC(=O)C(=O)[C@H](CC1CC1)NC(=O)[C@H](Cc1cc(F)c(O)c(F)c1)N[C@@H](c1cccc(F)c1)C(F)(F)F ; CC[C@H](NC(=O)[C@H](Cc1cc(F)c(O)c(F)c1)N[C@@H](c1ccc(F)cc1)C(F)(F)F)C(=O)C(=O)NC1CC1 ; CCCNC(=O)C(=O)[C@H](CC1CCC1)NC(=O)[C@H](Cc1cc(F)c(O)c(F)c1)N[C@@H](c1cccc(-c2nc(C)no2)c1)C(F)(F)F ; CCC[C@H](NC(=O)[C@H](Cc1cc(F)c(O)c(F)c1)N[C@@H](c1ccc(C2CCOCC2)cc1)C(F)(F)F)C(=O)C(=O)NC1CCCC1] 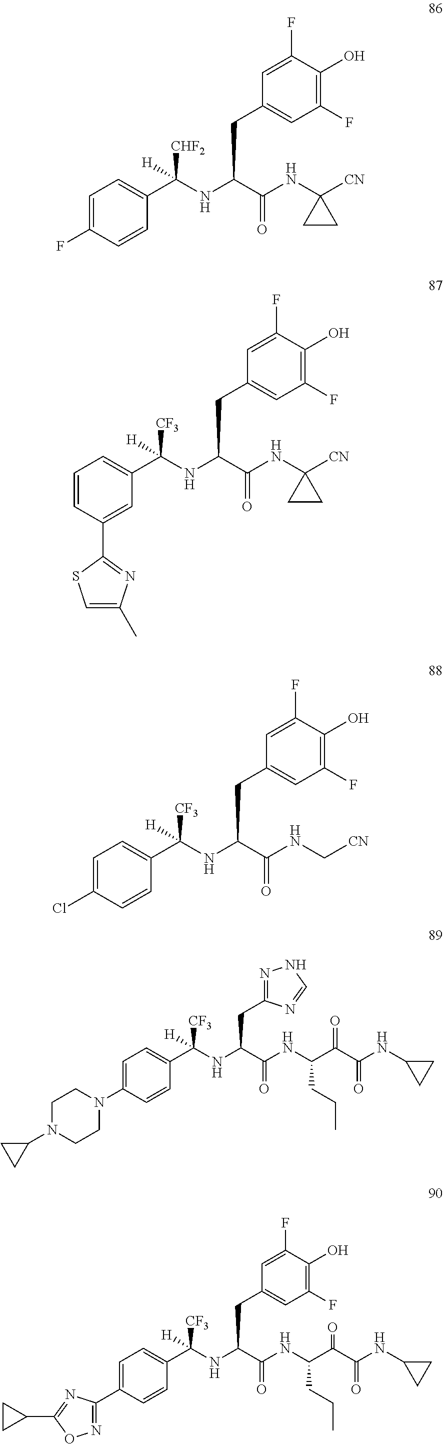 [SMILES: N#CC1(NC(=O)[C@H](Cc2cc(F)c(O)c(F)c2)N[C@@H](c2ccc(F)cc2)C(F)F)CC1 ; Cc1csc(-c2cccc([C@H](N[C@@H](Cc3cc(F)c(O)c(F)c3)C(=O)NC3(C#N)CC3)C(F)(F)F)c2)n1 ; N#CCNC(=O)[C@H](Cc1cc(F)c(O)c(F)c1)N[C@@H](c1ccc(Cl)cc1)C(F)(F)F ; CCC[C@H](NC(=O)[C@H](Cc1nc[nH]n1)N[C@@H](c1ccc(N2CCN(C3CC3)CC2)cc1)C(F)(F)F)C(=O)C(=O)NC1CC1 ; CCC[C@H](NC(=O)[C@H](Cc1cc(F)c(O)c(F)c1)N[C@@H](c1ccc(-c2noc(C3CC3)n2)cc1)C(F)(F)F)C(=O)C(=O)NC1CC1] 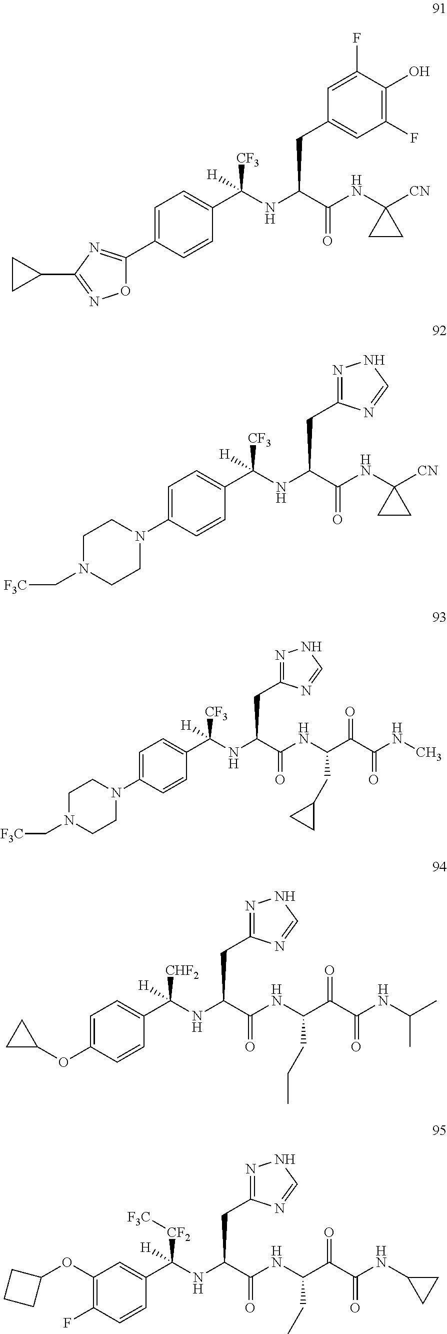 [SMILES: N#CC1(NC(=O)[C@H](Cc2cc(F)c(O)c(F)c2)N[C@@H](c2ccc(-c3nc(C4CC4)no3)cc2)C(F)(F)F)CC1 ; N#CC1(NC(=O)[C@H](Cc2nc[nH]n2)N[C@@H](c2ccc(N3CCN(CC(F)(F)F)CC3)cc2)C(F)(F)F)CC1 ; CNC(=O)C(=O)[C@H](CC1CC1)NC(=O)[C@H](Cc1nc[nH]n1)N[C@@H](c1ccc(N2CCN(CC(F)(F)F)CC2)cc1)C(F)(F)F ; CCC[C@H](NC(=O)[C@H](Cc1nc[nH]n1)N[C@@H](c1ccc(OC2CC2)cc1)C(F)F)C(=O)C(=O)NC(C)C ; CC[C@H](NC(=O)[C@H](Cc1nc[nH]n1)N[C@@H](c1ccc(F)c(OC2CCC2)c1)C(F)(F)C(F)(F)F)C(=O)C(=O)NC1CC1] 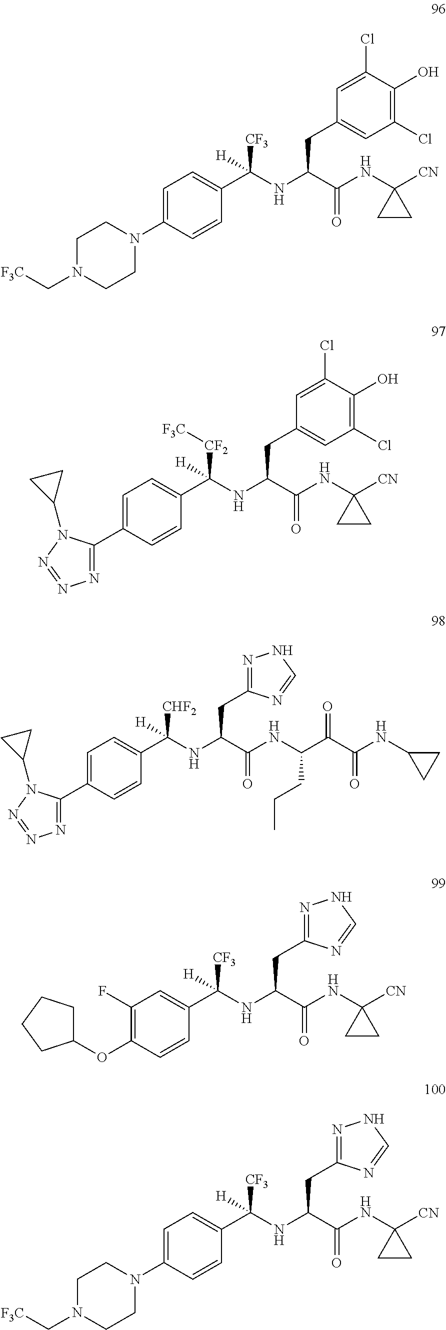 [SMILES: N#CC1(NC(=O)[C@H](Cc2cc(Cl)c(O)c(Cl)c2)N[C@@H](c2ccc(N3CCN(CC(F)(F)F)CC3)cc2)C(F)(F)F)CC1 ; N#CC1(NC(=O)[C@H](Cc2cc(Cl)c(O)c(Cl)c2)N[C@@H](c2ccc(-c3nnnn3C3CC3)cc2)C(F)(F)C(F)(F)F)CC1 ; CCC[C@H](NC(=O)[C@H](Cc1nc[nH]n1)N[C@@H](c1ccc(-c2nnnn2C2CC2)cc1)C(F)F)C(=O)C(=O)NC1CC1 ; N#CC1(NC(=O)[C@H](Cc2nc[nH]n2)N[C@@H](c2ccc(OC3CCCC3)c(F)c2)C(F)(F)F)CC1 ; N#CC1(NC(=O)[C@H](Cc2nc[nH]n2)N[C@@H](c2ccc(N3CCN(CC(F)(F)F)CC3)cc2)C(F)(F)F)CC1] 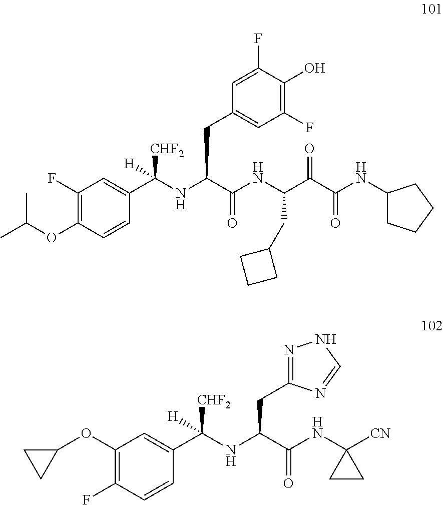 [SMILES: CC(C)Oc1ccc([C@H](N[C@@H](Cc2cc(F)c(O)c(F)c2)C(=O)N[C@@H](CC2CCC2)C(=O)C(=O)NC2CCCC2)C(F)F)cc1F ; N#CC1(NC(=O)[C@H](Cc2nc[nH]n2)N[C@@H](c2ccc(F)c(OC3CC3)c2)C(F)F)CC1]
and the pharmaceutically acceptable salts thereof.

19. The compound of claim 18 selected from Formulae 1-32, 49-64, 81-88, 90, 91, 96, 97 or 101.

20. The method of claim 3, wherein the cysteine protease is cathepsin B.
